SEQUENCE LISTING (29 sequences)

SEQ ID NO: 1 - RNA, 32 nt, synthetic construct
  source 1..32: mol_type = other RNA
aagcagtggtatcaacgcagagtacatrgrgg

SEQ ID NO: 2 - DNA, 57 bp, synthetic construct
  source 1..57: mol_type = other DNA
aagcagtggtatcaacgcagagtacttttttttttttttttttttttttttttttvn

SEQ ID NO: 3 - DNA, 66 bp, synthetic construct
  source 1..66: mol_type = other DNA
aatgatacggcgaccaccgagatctacacgcctgtccgcggaagcagtggtatcaacgcagagtac

SEQ ID NO: 4 - DNA, 33 bp, synthetic construct
  source 1..33: mol_type = other DNA
aagcagtggtatcaacgcagagtgaatrgrgrg

SEQ ID NO: 5 - DNA, 23 bp, synthetic construct
  source 1..23: mol_type = other DNA
aagcagtggtatcaacgcagagt

SEQ ID NO: 6 - DNA, 35 bp, synthetic construct
  source 1..35: mol_type = other DNA
gtctcgtgggctcggagagtgtgtataagagacag

SEQ ID NO: 7 - DNA, 33 bp, synthetic construct
  source 1..33: mol_type = other DNA
tcgtcggcagcgtcagatgtgtataagagacag

SEQ ID NO: 8 - DNA, 47 bp, synthetic construct
  source 1..47: mol_type = other DNA
caagcagaagacggcatacgagatnnnnnnnngtctcgtgggctcgg

SEQ ID NO: 9 - DNA, 48 bp, synthetic construct
  source 1..48: mol_type = other DNA
aatgatacggcgaccaccgagatctacacnnnnntcgtcggcagcgtc

SEQ ID NO: 10 - DNA, 29 bp, synthetic construct
  source 1..29: mol_type = other DNA
aatgatacggcgaccaccgagatctacac

SEQ ID NO: 11 - DNA, 90 bp, synthetic construct
  source 1..90: mol_type = other DNA
tcgtcggcagcgtcagatgtgtataagagacagnnnnnnnnnnnnnnnnnnnnvvvvvttttttttttttttttttttttttttttttvn

SEQ ID NO: 12 - DNA, 10 bp, synthetic construct
  source 1..10: mol_type = other DNA
tacctctcgg

SEQ ID NO: 13 - DNA, 10 bp, synthetic construct
  source 1..10: mol_type = other DNA
ctgccatcac

SEQ ID NO: 14 - DNA, 10 bp, synthetic construct
  source 1..10: mol_type = other DNA
aactcgttcg

SEQ ID NO: 15 - DNA, 10 bp, synthetic construct
  source 1..10: mol_type = other DNA
taagtgctcg

SEQ ID NO: 16 - DNA, 10 bp, synthetic construct
  source 1..10: mol_type = other DNA
ccatatcgga

SEQ ID NO: 17 - DNA, 10 bp, synthetic construct
  source 1..10: mol_type = other DNA
ctaacacaga

SEQ ID NO: 18 - DNA, 10 bp, synthetic construct
  source 1..10: mol_type = other DNA
cattgcctgg

SEQ ID NO: 19 - DNA, 10 bp, synthetic construct
  source 1..10: mol_type = other DNA
aatgagagag

SEQ ID NO: 20 - DNA, 10 bp, synthetic construct
  source 1..10: mol_type = other DNA
ttccaatacg

SEQ ID NO: 21 - DNA, 10 bp, synthetic construct
  source 1..10: mol_type = other DNA
acagttagca

SEQ ID NO: 22 - DNA, 10 bp, synthetic construct
  source 1..10: mol_type = other DNA
accaatgcgg

SEQ ID NO: 23 - DNA, 10 bp, synthetic construct
  source 1..10: mol_type = other DNA
agatccgctc

SEQ ID NO: 24 - DNA, 10 bp, synthetic construct
  source 1..10: mol_type = other DNA
attcgcgtcg

SEQ ID NO: 25 - DNA, 10 bp, synthetic construct
  source 1..10: mol_type = other DNA
cacaccgaga

SEQ ID NO: 26 - DNA, 10 bp, synthetic construct
  source 1..10: mol_type = other DNA
cgatgcacac

SEQ ID NO: 27 - DNA, 60 bp, synthetic construct
  source 1..60: mol_type = other DNA
aatgatacggcgaccaccgagatctacacnnnnnnnnnntcgtcggcagcgtcagatgtg

SEQ ID NO: 28 - DNA, 71 bp, synthetic construct
  source 1..71: mol_type = other DNA
cgtcagatgtgtataagagacagnnnnnnnnnnnnnvvvttttttttttttttttttttttttttttttvn

SEQ ID NO: 29 - DNA, 70 bp, synthetic construct
  source 1..70: mol_type = other DNA
aatgatacggcgaccaccgagatctacacnnnnnnnntcgtcggcagcgtcagatgtgtataagagacag

(19) United States
(12) Patent Application Publication
(10) Pub. No.: US 2024/0401026 A1
(43) Pub. Date: Dec. 5, 2024

(54) SYSTEM AND METHOD FOR SINGLE CELL PHENOTYPICAL PROFILING AND DETERMINISTIC NANOLITER-DROPLET ENCAPSULATION AND DETERMINISTIC DROPLET CONSORTIA ASSEMBLIES

(71) Applicant: ECOLE POLYTECHNIQUE FEDERALE DE LAUSANNE (EPFL), Lausanne (CH)

(72) Inventors: Johannes BUES, VILLARS-SOUS-YENS (CH); Jörn PEZOLDT, LAUSANNE (CH); Bart DEPLANCKE, CUGY (CH)

(21) Appl. No.: 18/691,308

(22) PCT Filed: Sep. 12, 2022
(86) PCT No.: PCT/IB2022/058573

§ 371 Date: Mar. 12, 2024

(30) Foreign Application Priority Data
Sep. 13, 2021 (WO) PCT/IB2021/058310

(51) Int. Cl. C12N 15/10; C12M 1/32; C12M 1/34; C12M 3/06

(52) U.S. Cl. CPC C12N 15/1065; C12M 23/12; C12M 23/16; C12M 41/46; C12N 15/1075

(57) ABSTRACT

The present invention concerns a system for phenotypical profiling of at least one object and deterministic nanoliter-droplet encapsulation, comprising sample supplying means, buffer supplying means; a microfluidic chip comprising an encapsulation area or structure in which the object is encapsulated with a quantity of the reaction buffer by the droplet; detection means configured to detect the passage of the object through the first imaging chamber; at least one valve configured to stop the flow of the sample buffer when the detection means detect passage of the object through the first imaging chamber; phenotypical assessing means configured to assess the phenotype of the object when the flow of the sample buffer is stopped by the valve and the object is at an object stopping site; a droplet deposition means configured to deposit the droplet in a well or in a well of a multi-well plate and comprising an outlet capillary.

### CROSS-REFERENCE TO RELATED APPLICATIONS

The present application claims priority to international PCT application number PCT/IB2021/059310 filed on Sep. 13, 2021, the entire contents thereof being herewith incorporated by reference.

### FILED OF THE INVENTION

The present disclosure relates to a device or system for high throughput single-cell studies and more particularly to a system and a method for single cell phenotypical profiling and deterministic nanoliter-droplet encapsulation and deterministic droplet consortia assemblies.

### BACKGROUND

Molecular profiling methods utilizing next generation sequencing dissect transcriptomic, genomic and epigenomic features of biological specimen. They are the methods of choice to delineate cellular states, individual responses, and pathologic aberrations, and drive the discovery of biomarkers across pathologies and the identification of therapeutic targets of drugs.

Particularly the “transcriptome” as the entity of all RNAs transcribed by a defined tissue, biopsy, or a single cell, reveals not only the metabolic state of the cell, but also functionally relevant mutations.

Currently, high-throughput RNA-sequencing (RNA-seq) is replacing targeted and microarray approaches to enable comprehensive biological, medical, clinical and drug research.

Improvements in samples processing for RNA-seq have been closely followed by an expanding methods portfolio for profiling of single cells; so called scRNA-seq, which has revealed the vast heterogeneity across cellular responses.

To probe functional properties of same heterogeneity in the context of biomarker discovery, diagnostics and treatment regimes, transcriptional profiling needs to be implementable at a larger scale, lower costs and with higher reproducibility.

To achieve these goals, technologies are required to reduce manual labor & consumables, to flexibly integrate any molecular profiling approach, to be highly automated and to enable the acquisition of functional phenotypes together with the metabolic state of the cell.

### Single Cell Profiling

The first single-cell RNA-sequencing (scRNA-seq) experiments of Tang et al. built upon existing well-based methods¹ and were consequently limited in throughput. By implementing basic multiplexing and miniaturization,²⁻⁴ throughput was increased, compatible with most cell isolation techniques attained⁵ and scRNA-seq profiling could be coupled to complementary measurements, such as imaging.⁶ Despite the miniaturization and automation, costs remained high on a per cell basis (>5 CHF) due to the large reaction volumes. Consequently, most scRNA-seq experiments are currently carried out using high-throughput droplet-based⁷⁻⁹ or nanowell-based¹⁰⁻¹² platforms.

Both droplet- and nanowell-based approaches utilize highly parallelized cell capture and low reaction volumes, reducing costs per cell by an order of magnitude (<0.5 CHF) and making processing of thousands of cells per sample routinely possible. Briefly, in order to generate a gene expression profile from a cell in a droplet the cell is paired with a mRNA capture bead inside a nanoliter water-in-oil droplet. Droplets are assembled stochastically in microfluidic devices by co-flowing a bead containing solution with a cell suspension. Inside the droplet, mRNA is liberated from the cell (cytoplasm/nucleus) by lysing the cell, and the released mRNA is captured by oligonucleotides present on/within the mRNA capture bead. To discriminate mRNA molecules from different cells, the oligonucleotides of each bead contain a common nucleotide sequence, here termed cell barcode, that is specific and unique to each bead. During reverse transcription, this sequence is incorporated in the cDNA molecules generated from the cell's mRNA.

The afore-described droplet-based process entails two stochastic steps: the uncontrolled cell-capture process and the unknown barcode sequence of the mRNA capture bead during encapsulation. This results in capability limitations of same droplet-based assays, being: 1) the stochastic cell capture making it impossible to select for or discard cells and resulting in a majority of faulty encapsulation (e.g. empty droplets or cell doublets¹³⁻¹⁵), and 2) the inability to acquire qualitatively high-level phenotypical profiles of molecularly profiled cells. Furthermore, as the production of functionalized particles is challenging, the reliance on beads in these systems introduces additional restrictions.

This hampers the establishment of new or improved molecular profiling protocols that require different types of beads. Furthermore, droplet-based systems have displayed inferior sensitivity and quality consistency¹⁶ which most likely can be attributed to challenges associated with production of high-quality beads.

Therefore, a substantial proportion of technology development is dedicated to adapt available droplet-based strategies, relying on mRNA capture beads, to overcome specific drawbacks, but have so far not yielded a next level evolutionary step towards a holistic and deterministic cellular profiling. These partial solutions include, bioinformatics tools for identification of failed encapsulation events for droplet-based systems¹³⁻¹⁵, cell labeling strategies to increase sample numbers per run,^{17,18} and DNA-barcode labeled antibodies enabling detection of surface markers yielding basic phenotypical profiles.¹⁹ Bead-based nanowell systems were also augmented with a deterministic barcoding scheme to allow for imaging.^{20,21} Nevertheless, even with added layers of complexity, neither conventional nor specialized high-throughput approaches were capable of reaching the flexibility of plate-based approaches particularly in the context of droplet-based approaches. Overall, this has led to a situation in which two experimental concepts exist, flexible precision assays in wells, which are costly, and inflexible high-throughput platforms.

In order to bridge the gap between these two setups, a technology is required that enables to encapsulate cells deterministically in a nanoliter-sized droplet with liquid biochemistry of choice yielding the ability to track the encapsulation event via a known integrated barcode.

### Cell Population Profiling

Afore-mentioned inflexibilities in adapting biochemistries (or protocols) on high-throughput scRNA-seq platforms also limits translation of single-cell solutions into assays performed on bulk cells, for which similarly elaborate workflows for library preparation are required. Massive multiplexing during library indexing is well established, so called dual-indexing, for RNA-seq, ATAC-seq and ChIP-seq library preparation. All those processes entail execution of the whole library preparation workflow for each individual sample. Therefore, additional barcoding strategies were developed which allow sample barcoding at earlier processing steps, and thus allow sample pooling. In the context of RNA-seq, these methods commonly rely on 3′-counting of transcripts and have been adopted in several recent studies, including for example PLATE-seq,²² and BRB-seq²³ which rely on pre-isolation of mRNA.

Hence, a sample processing approach that would make mRNA isolation obsolete, would not only reduce costs but also permit to process more samples and samples with vastly lower cellular input material. This is precisely what some droplet-based scRNA-seq methods provide: direct in-droplet lysis and reverse transcription. Despite this advantage, the unknown barcodes embedded in the mRNA-capture beads, does not allow to pool several samples in one well, making it impossible to leverage the processing advantages of droplet-based methods into bulk molecular profiling applications.

The overarching goal of a next-level molecular profiling techniques would thus encompass to increase the throughput, elevate sensitivity, decrease reagent consumption and personnel time; all of which is feasible by combining the advantages of nanoliter-sized reaction volumes together with automated encapsulation of cellular particles with a deterministic barcoding system.

Deterministically barcoding cellular entities directly within nanoliter-droplets would enable efficiency benefits already at the initial step of sample handling and the involved resources, which in turn would multiply the leverage of downstream multiplexing approaches. Such a nanoliter-droplet-based approach would improve the following steps of common to all molecular profiling approaches: i) Cell quantification, ii) Lysis of cells/nuclei, iii) Extraction of RNA/DNA, iii) Quantification and normalization of RNA/DNA, iv) Barcoding of RNA molecules, v) Pooling of samples.

Each of these steps involves a substantial economical and experimental effort:
i) Cell quantification and phenotypical profiling is the very initial step previous to isolation of any given molecule. Most commercial kits are limited to optimal cell numbers and thus cell quantification needs to be carried out on a per sample basis. This process can be carried out manually using a Neubauer counting chamber (2 min per sample) or commercial solutions like Countess (1 CHF per sample), both of which apply to for example drug screens carried out in conventional multi-well devices. Assuming a standard screen to encompass 100 Samples this either amounts to 100 CHF personnel and/or consumable costs. If samples require sorting for rare cell populations (e.g. ex vivo studies), utilizing fluorescence activated cell sorting (FACS), costs are at least 10 to 20 fold higher, due to the trained operator and cost-intensive machines required.
ii) The extraction of RNA from cellular input material, such as a cell/nuclear lysate, is performed on a per sample basis, and is by that intrinsically labor-intensive; easily taking one day of manual labor with an additional average cost of 5 CHF per sample. Hence, amount to 500 CHF costs per 100 samples. The same applies for DNA for ChIP-seq or TF-seq assays.
iii) RNA quantity normalization across samples per pool is extremely critical to ensure that, subsequent to the initial barcoding, each of the samples is molecularly equally represented in the pooled library comprising or consisting for example 100 samples. This is important because if one sample is over-proportionally (e.g. 50 fold differences are common), it will take up 50% of the total sequencing space for the given pooled sequencing library, which leaves substantially less sequencing reads and therefore biological insights for the remaining 99 samples in the pool. The worst case being that the whole library preparation process, amounting to one day of labor and 50 to 200 CHF consumable costs per pooled library requires repetition. Therefore, RNA sample input normalization is extremely critical and requires a half-day of manual labor for initial concentration measurement, manual concentration adjustment and confirmation concentration measurement. The same applies for DNA for ChIP-seq or TF-seq assays.
iv) Barcoding of RNA during reverse transcription (RT) requires the pre-isolated RNA, barcoded DNA & unique molecular identifier-containing oligo-dTs (RT-primer), template switch oligos (TSO) or an additional second strand synthesis step and RT enzyme. During the enzymatic reaction, poly-adenylated RNA binds to the poly-dT tail of the barcoded DNA primer which serves as a primer for the RT enzyme for the first synthesis. RT-primers contain one unique barcode per sample. Therefore, each cDNA molecule from a given sample, contains the same barcode at the 5′end, with each molecule containing a unique molecular identifier (UMI). Additionally, the 3′end is labelled by the TSO sequence. Conventionally, these enzymatic reactions are carried out per sample in volumes of >5 μl. Particularly, high-quality RT enzymes are cost-intensive driving the price per reaction on average to 2 CHF per sample. Hence, the cost for reverse transcription including the additional consumables amounts to approximately 300 CHF per sample and involves a half day of manual labor.
v) Barcoding of DNA is carried out conventionally on samples that are pooled into one cDNA library subsequently to RT and then further processed within one tube. This already amounts to a substantial reduction in labor time in comparison to performing sequencing library preparation for each sample in a multi-well unit. The last step previous to sequencing usually involves either ultra-sonic shearing, but more commonly enzymatic tagmentation and adapter ligation amounting to 40 CHF per pooled library and a half day of manual labor for a pooled library of 100 samples.

In the case of DNA profiling methods such as approaches to assess transcription factor binding, histone modifications or simply chromatin accessibility, per sample costs due to the utilized enzymes and antibodies are similarly excessive and could be vastly reduced.

Overall, with the currently available methods, the processing of 100 samples amounts to on average 1000 CHF for consumable and three days of manual labor, yielding a pooled sequencing library. The process is to some extent scalable, but essentially any additional 100 samples will add at least one and a half days of manual labor (see i) & ii).

### SUMMARY

It is thus a goal of the present disclosure to address the above-mentioned inconveniencies.

It is one aspect of the present disclosure to provide a system according to claim 1 and a method according to claim 17. Further advantageous features can be found in the dependent claims.

Advantages of the system of claim 1 the ability to combine phenotypical profiling of single cells paired together with the molecular profiling. The direct profiling of these cell phenotype addressed with imaging and the molecular profiling is enabled by the ability of deterministically assembling droplet consortia in a multi-well format. Deterministic assembly of droplet consortia is achieved by a microfluidic system, designed to enable stopped-flow triggered upon machine-vision driven detection of entities on the microfluidic system at the phenotypical profiling/imaging site.

The above and other objects, features and advantages of the present disclosure and the manner of realizing them will become more apparent, and the disclosure itself will best be understood from a study of the following description with reference to the attached drawings showing some preferred embodiments of the disclosure.

### BRIEF DESCRIPTION OF THE DRAWINGS

The accompanying drawings, which are incorporated herein and constitute part of this specification, illustrate the presently preferred embodiments of the disclosure, and together with the general description given above and the detailed description given below, serve to explain features of the disclosure.

FIGS. 1A and 1B show an overview of a quantification/counting and automated microfluidic entity concentration adjustment. A) Entities are flown into the chip from a pressurized container 104, 102. Entities are imaged, blob detection applied, and the number of entities determined that pass through the “Imaging Volume” 101, 103 and concentration is calculated based on a pre-defined calibration curve or via event-based sensing. Dilution buffer, also obtained from a pressurized container 105, 107 is co-ejected 106 with the entity-containing buffer. The desired concentration of the entities 108 is achieved by adjusting the pressure within the dilution buffer chamber and the container holding the entity solution.

B) Entities are fed into the chip from a pressurized container. Entities are imaged in the “Primary counting Chamber” 103, blob detection applied, and the number of entities determined that pass through the “Primary counting Chamber” and concentration is calculated based on a pre-defined calibration curve or via event-based sensing. Dilution buffer, also obtained from a pressurized container is co-flown with the entity-containing buffer and the dilution of entities 109 is quantified in the “Secondary Counting Chamber” 111, using the same microscopy objective. By applying dual quantification of the entities the dilution can be adjusted rapidly previous to entering the chip.

FIGS. 2A to 2F show an embodiment of an automated entity/reaction buffer supply and capillary handling for droplet deposition 200. A) In order to supply and exchange liquids running on the microfluidic system a device was developed. Vessels 203 containing entity/reactant solution are placed in a wheel like structure 205 that can be rotated (round arrow). This wheel is connected to a linear rail 204 and a pneumatic cylinder 201 which enables to insert/retract the capillary 202 that is in a fixed position. B) The capillary is held in a structure that allows for pressurization of the vessel. C) In order to inject fluids into the system the vessel is pressurized utilizing 207 the above-described pressure regulator. The vessel 203 is sealed from the ambient air with a rubber gasket 208 that is pressed onto the vessel by force generated by the pneumatic cylinder. The air-pressure in the vessel drives the fluid 209 into the capillary which is connected to the microfluidic system 206. D) The outlet capillary guiding the produced droplets to a multiwell plate is spatially coordinated by an XY-stage 214. Z-positioning 211 of the capillary is achieved by either one of two mechanisms: E) The first mechanism utilizes a capillary holder that is connected to a pneumatic cylinder 210, 211 allowing for movement of the capillary. F) The secondary mechanism repositions the multiwell plate while the capillary stays fixed in space 211, 212, 214.

FIG. 3A to 3G depict a process, efficiencies for deterministic droplet scRNA-seq and exemplary microfluidic designs for deterministic droplet assemblies. A) Conceptual overview of the microfluidic chip. Similar as for deterministic co-encapsulation, the chip is comprised of inlets for oil 303a, cells 301, and RT solution 302, and outlets for waste liquid 304 and for the generated droplets 305. Except for the droplet outlet section, each inlet and outlet was added with an on-chip valve (numbered boxes; 1: cell valve 322, 2: primary reaction buffer solution valve, 4: oil valve, 5: waste valve 323, 6: sample valve), and one additional valve was integrated for dropleting (3: dropleting valve). Downstream of the sample valve, an additional oil inlet 307, 308 controlled by an off-chip solenoid was integrated, that is used for flushing droplets through a capillary into the processing vessels (shown in C). B) On this chip a 3 step processes was implemented to 1) stop a cell 309, 2. pair the cell with RT solution at the dropleting point 310, and 3) encapsulation of the cell in a droplet by actuation of the dropleting valve 311. C) Upon droplet generation the droplet is 4.) the isolated from encapsulation area and loaded into the outlet port where it is 312 5.) flushed into the outlet vessel 313. D) Cell positioning by valve over pressurization. A cell is getting detected in the detection ROI 314, and the valve is closed. Next, the inlet pressure is increased to induce a valve leak 316, moving the cell towards the stop ROI 315. Once in the stop ROI, the pressure is decreased, thereby stopping the cell in a defined position. E) Encapsulation efficacies for different cell types (HEK 293T, RAW 264.7, embryonic stem cells (ES)) and ex vivo isolated murine neural nuclei. Encapsulation efficiency corresponds to the number of entities detected in the detection ROI and successfully stopped and encapsulated in a nanoliter-droplet. n=6-10 replicates across 96 well. F) Cellular entities are loaded to the “Holding Chamber” or first imaging chamber 319 via the “Cellular Entity Inlet” 318 using an off-chip valve. From the “Holding Chamber” cells are flow toward the encapsulation 320 point via high-precision pressurization of the channel carrying the “Cellular Entity Running Buffer”. Upon detection via machine-vision the cellular entity is encapsulated in a nanoliter-droplet together with the “Reaction Buffer” from the primary reaction buffer inlet. G) An entity is flown on the microfluidics device via a microfluidic sample inlet channel or capillary 318 and stopped at the first imaging chamber and/or detection site and/or phenotypical assessment site in the first imaging chamber 319 subsequent to the capillary or microfluidic inlet for placement buffer 317. The entity is displaced towards the secondary Imaging chamber 321, by liquid flow from the capillary or microfluidic inlet for placement buffer 317. The entity is encapsulated with primary reaction buffer from the primary reaction buffer inlet 302 and a plug is formed at the encapsulation area 320. The droplet is formed by shearing the plug with droplet-forming oil from the droplet forming oil inlet 307.

FIGS. 4A to 4D show an embodiment of the detection, deterministic stopping and focal depth imaging of cells. A) Schematic illustration of the epi-fluorescence microscope with integrated bright-field illumination and electrical tunable lens, enabling detection, deterministic stopping and high-resolution imaging of an entity 401, 402, 403, 404, 405, 406, 407, 408, 409, 410, 411, 412, 413, 414, 415, 416, 417, 418, 419, 420, 421, 422, 423, 424, 425, 426, 427, 428. B) Schematic overview of the control system with a processor 428. C) Multiple channels of the microfluidics chip are visible. Left-to-Right: For fast and accurate detection, a large window in the wide channel is chosen. After cell detection, the cell is placed in the imaging ROI. The target cell is marked by a yellow arrow. D) Images of a HeLa-cell in the ROI taken with different ETL settings changing the optical power from −1.5 to +3.5 diopters in 0.83 diopter steps.

FIGS. 5A and 5B depict an embodiment of a scaffold for integrating deterministic automated deposition of encapsulated entities. A) Rendering of the Computer-aided Design (CAD) Model of the chip-to-world scaffold developed for the system. The chip 408 is oriented vertically and monitored by a horizontal microscope 101. The position capillary connected to the chip 506 is held by a pneumatic actuation mechanism that allows for repositioning along the Z-axis 507. Located beneath the chip is a XY-stage 505 holding a 96-well plate. 213 B) Each well of the plate is loaded with one droplet by moving the outlet capillary from well to well 508, 509. C) Shows the top-view on a PCR tube loaded with 10 sample droplets 510 (blue), surrounded by stuffer droplets (clear).

FIG. 6 Device for establishing electric field metal plates 602 placed adjacent to each row of a multi-well plate 213.

FIGS. 7A to 7E show results regarding the use of stuffer-droplets serve as storage containers for small molecules and enzymatic reactions. A) Five HEK293T cells were encapsulated with reverse transcription buffer reaction mix in reaction droplets and deposited in one well of a 96 well plate, containing Stuffer-droplets with varying dNTP concentrations. Reverse transcription, well-coding and PCR amplification were carried out and cDNA concentration measured using Qbit (n=4). B) Fragment analyzer size distribution profile for exemplary sample at 2 mM dNTP concentration in Stuffer-droplets. C) 10 or 100 HEK293T cells were encapsulated with reaction buffer containing Triton X 100 and Proteinase K. Subsequent to cell lysis, reaction droplets were merged with Stuffer-droplets containing Tn5 loaded with adapters amenable to sample-indexing via overhang-PCR. Size distribution was obtained via Fragment analyzer. D) Representative karyotype assessments of single cells from peripheral blood mononuclear cells of patient-derived acute myeoloid leukemia.

FIGS. 8A to 8D depict results of the implementation of different RNA-seq biochemistries in nanoliter-droplets. A) Using IRIS 10 single HEK 293T were encapsulated in individual nanoliter-droplets containing Smart-seq v2 biochemistry and deposited in one unit of a multi-well plate. RT was performed in droplets and cDNA amplified via PCR for 23 cycles. cDNA content per 10 cells was determined using Qbit and size distribution assessed via FragmentAnalyzer. B) cDNA size distribution profile of 10 HEK 293T encapsulated in individual nanoliter-droplets containing SCRB/BRB-seq biochemistry. C) Barnyard plot for Species mixing experiment of indicated numbers of HEK 293T (human) or RAW 264.7 (mouse) cells singly encapsulated with distinct barcodes per species process using SCRB/BRB-seq biochemistry. D) Percent of UMIs not mismatching with the designated barcode identifying the species from experiment performed in (C).

FIGS. 9A to 9C concern result of multi-indexing of cells via deterministic barcoding via cell, well and plate code. Dual barcoding chemistry for scRNA-seq in droplet consortia. A) RT was executed in droplet consortia, adding the cell code to the cDNA. Next, droplets were merged, and PCR performed, adding the well code index. Finally, all material was pooled, purified, and the plate code added after Tn5 tagmentation. B) Distribution of mapped reads per cell, well, and plate barcode. C) Distribution of UMI counts per cell, well, and plate barcode.

FIGS. 10A to 10D show results of multiplexed RAW macrophage LPS stimulation experiment. A) RAW 264.7 cells were either treated with 100 ng/ml LPS (+LPS) or left untreated (Ctrl). Ctrl and LPS cells were introduced on the system in an alternating fashion, associating cell barcodes to specific samples. B) UMAP embedding of all collected cells. Cells are colored by their cluster association. C) Heatmap of differentially expressed genes of both clusters. D) UMAP embeddings of cells per defined cell barcodes.

FIG. 11 shows an example of a multi-indexing for scATAC-seq. Loading of nuclei is executed in bulk and does not add the entity-specific oligos to the DNA. Entities are singly encapsulated in a nanoliter-droplet containing PCR enzyme, buffer and barcoded oligos for overhang PCR. Multiple droplets containing a single entity, each of which contains a unique barcode identical for each well of the multi-well plate, are assembled to droplet consortia per unit of multi-well plate. PCR reaction is performed appending the barcoded oligo to the tagmented DNA. Next, droplets are merged, and a secondary PCR performed, adding the well code index. Finally, all material was pooled, purified, and the plate code added after Tn5 tagmentation.

FIGS. 12A to 12D show results regarding the use of multi-indexing to perform 3′RNA-seq on multi-droplet assemblies. HEK 293T were singly encapsulated in nanoliter-droplets containing reaction buffer with 3 or 5% of polyethylene glycol (PEG) for molecular crowding. Control (Ctrl) samples only contained the reaction buffer. Five cells were deposited per well of the multi-well plate and RT performed in droplets. Droplets were merged and well-barcode (WC) was appended via an overhang PCR. Plate-barcode (PC) was appended via tagmentation. RT was performed with 3% PEG for PC1&2, 5% PEG for PC3&4 or 0% PEG for PC5/6 (Ctrl). Each PC contains eight WCs. A) Reads mapping to the human genome for each of the eight WCs per PC. B) UMIs per WC for each of the six experimental conditions. C) Percentage of UMIs per gene for the Top 30 represented genes across all replicates. D) Percentage of reads for mitochondrial, ribosomal, and heat-shock proteins across all replicates and experimental conditions.

Herein, identical reference numerals are used, where possible, to designate identical elements that are common to the figures. Also, the images are simplified for illustration purposes and may not be depicted to scale.

### DETAILED DESCRIPTION OF SEVERAL EMBODIMENTS

### Numerical References Used in the Drawings

101—Detection means and/or phenotypical assessing means or Imaging objective for machine vision
102—Entity sample port
103—Volume for counting of entities or first counting chamber
104—Sample buffer capillary
105—Dilution buffer capillary
106—Intersection dilution and sample buffer
107—Dilution buffer inlet
108—Diluted entities
109—Intersection dilution buffer and entity buffer
111—Secondary counting chamber
200—Sample supplying means and/or Buffer supplying means
201—Pneumatic cylinder
202—Capillary to microfluidics
203—Vessel
204—Linear rail
205—Rotating wheel
206—Solution/Buffer to microfluidic chip
207—Air pressure from pressure regulator
208—Gasket
209—Buffer/Sample/Solution reservoir
210—Capillary holder
211—Pneumatic cylinder
212—Capillary from microfluidics
213—droplet storing vessel such as well or a well of a multi-well plate
214—Base connected to XY-stage
215—XY-stage
301—object or entity or cellular entity
302—Capillary or microfluidic channel primary reaction buffer inlet
303a—droplet forming substance inlet
303b—droplet formation microchannel or droplet forming substance microchannel
304—waste or discarding outlet
305—droplet outlet capillary
307—Microfluidic channel or capillary providing droplet supporting oil or mineral oil or other oil
308—off-chip valve
309—stop cell step
310—co-expulse primary reaction buffer and entity step
311—encapsulation step
312—droplet isolation step
313—flush droplet step
314—detection region of interest step
315—stop region of interest step
316—leak flow step
317—Capillary or microfluidic inlet for placement buffer or first reaction buffer
318a—First microfluidic channel or Microfluidic sample inlet channel or capillary
318b—Second microfluidic channel
319—First imaging chamber and/or detection site and/or phenotypical assessment site
319a—Object stopping site
320—Encapsulation area
321—Secondary Imaging chamber
321a—Object stopping site, for example in the secondary imaging chamber
322—Sample buffer stopping flow valve
323—Discarding valve
401—Arduino board
402—(U96) FPGA
403—White LED
404—Fiber-coupled light
405—Detection camera
406—(805 nm) LED
407—(805 nm) long-pass filter
408—Microfluidic chip
409—Microscope objective
410—(805 nm) short-pass filter
411—Multi band pass filter
412—tube lense (focal length 200 mm)
413—4f-relay
414—(Silver) mirror
415—lens (focal length 100 mm)
416—electrical tunable lens
417—lens (focal length 100 mm)
418—(2×) beam expander
419—Imaging camera
420—User interface
421—Laser diode controller
422—Laser diode (637 nm)
423—Laser diode (488 nm)
424—Laser diode (405 nm)
425—(650 nm) long-pass mirror
426—(505 nm) long-pass mirror
427—(420 nm) long-pass mirror
428—Processors
505—Multi-well plate handler or droplet deposition means
506—Capillary handler or outlet capillary
507—Pneumatic actuation mechanism or droplet deposition means
508—Process of loading a droplet
509—Process of moving the droplet deposition capillary
510—Droplet containing an object or a cellular entity and primary reaction buffer
602—Slots for electric-field insulated metal plates with alternating current attached at alternating positions

The present disclosure concerns a system for phenotypical profiling of at least one object and deterministic nanoliter-droplet encapsulation of same at least one object. The components of the system according to the present disclosure are illustrated for example in FIGS. 2A to 2F, FIGS. 3A to 3G, FIGS. 4A to 4B, FIGS. 5A to 5C.

The at least one object may be a cell or a cellular entity or a cell derived entity, a bacteria, a microsphere, a protozoan, an algae, a spore, a molecule, an enzyme, a microparticle, a vesicle, a microvesicle or a microorganism.

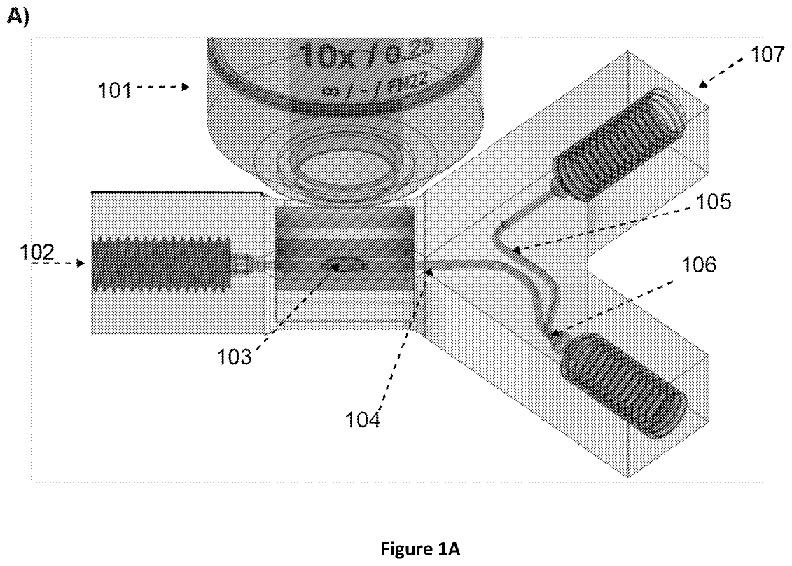

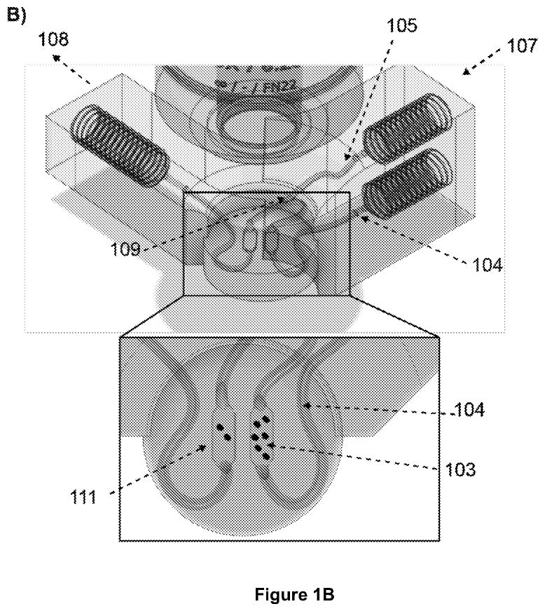

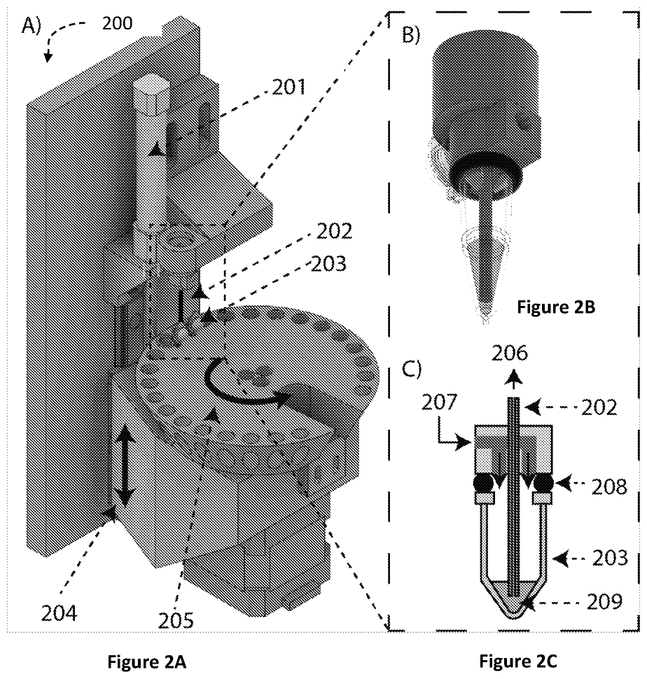

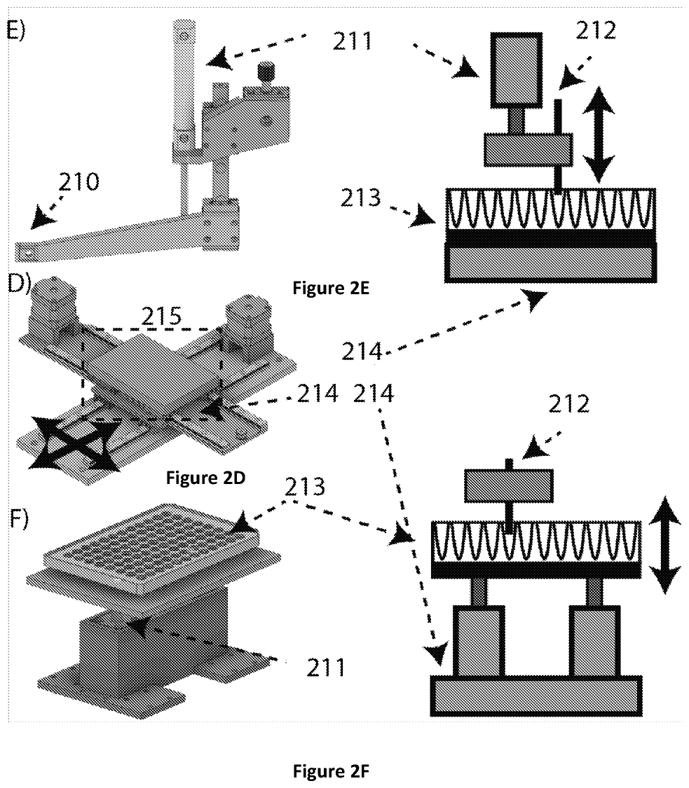

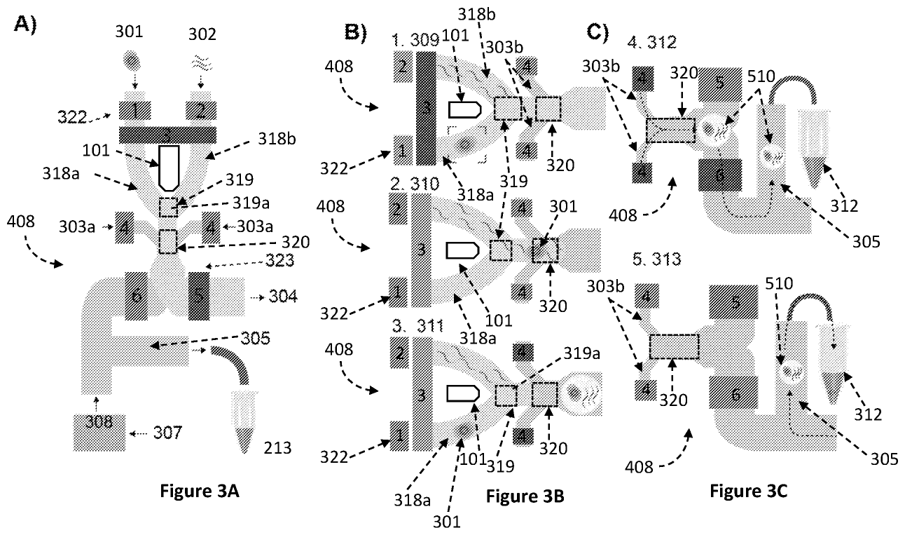

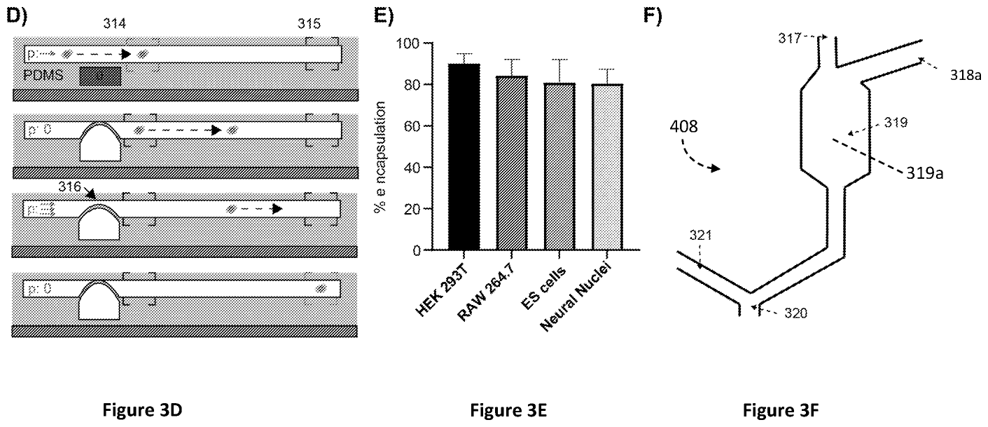

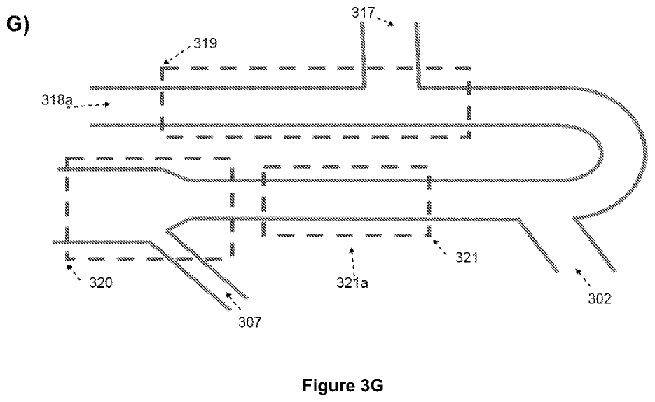

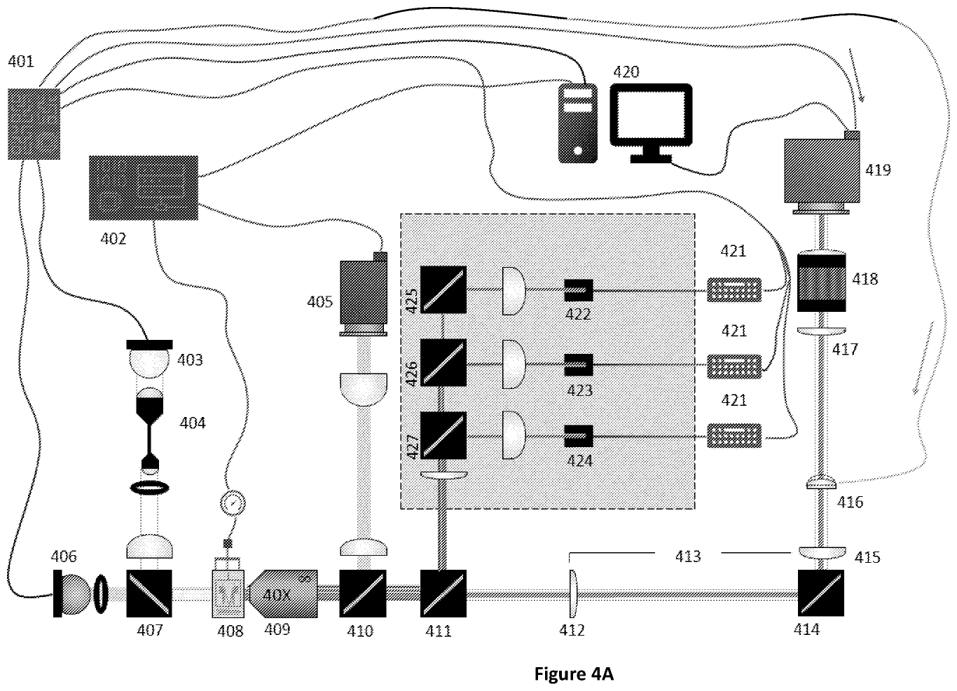

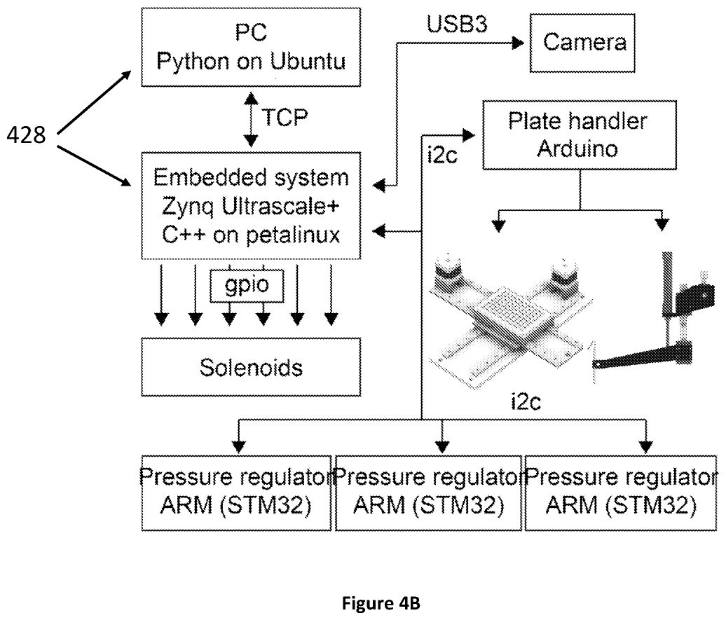

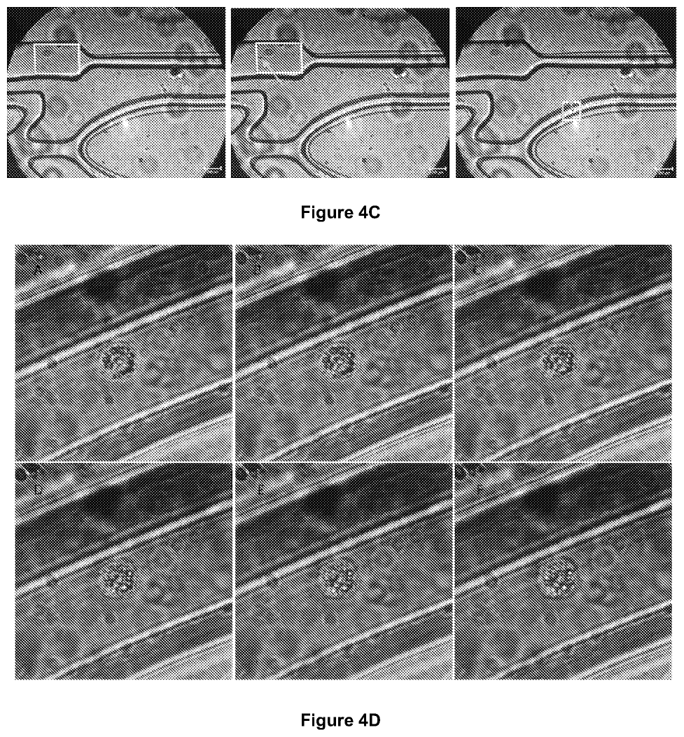

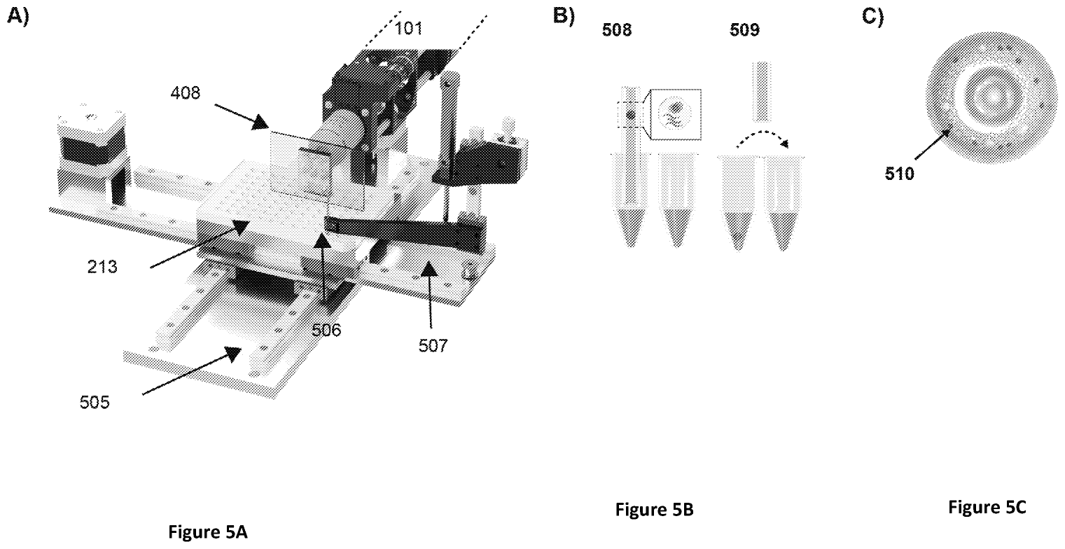

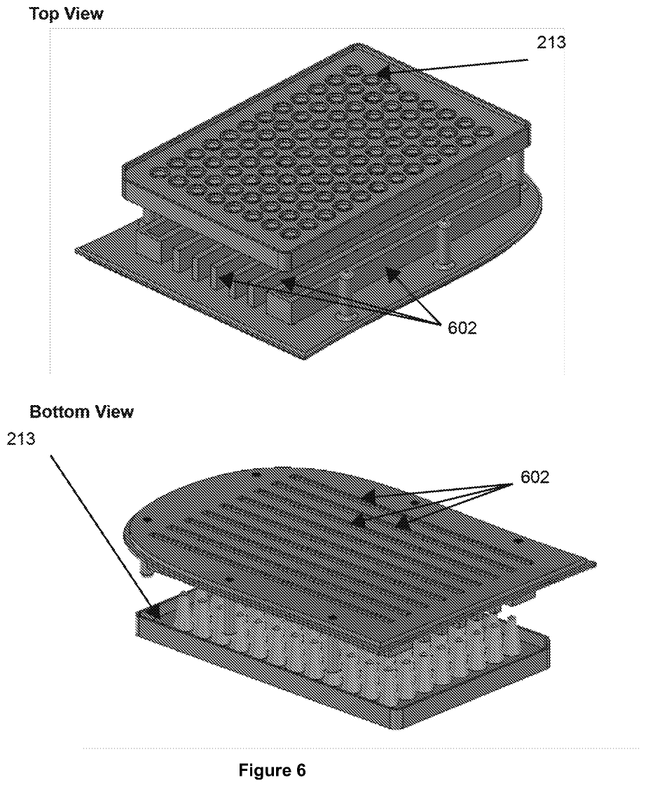

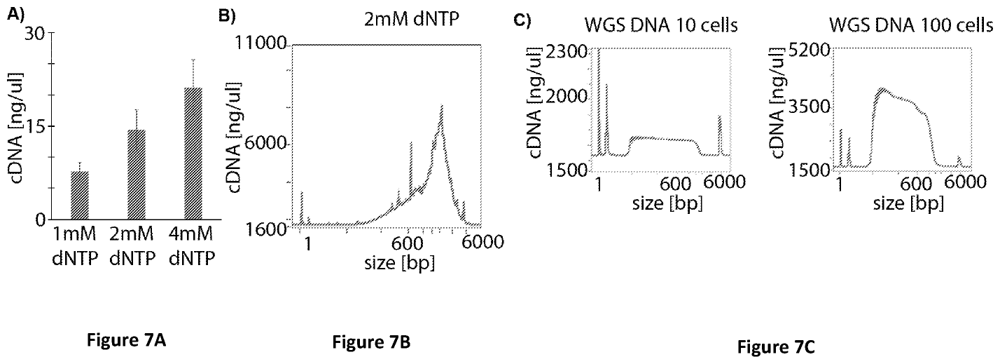

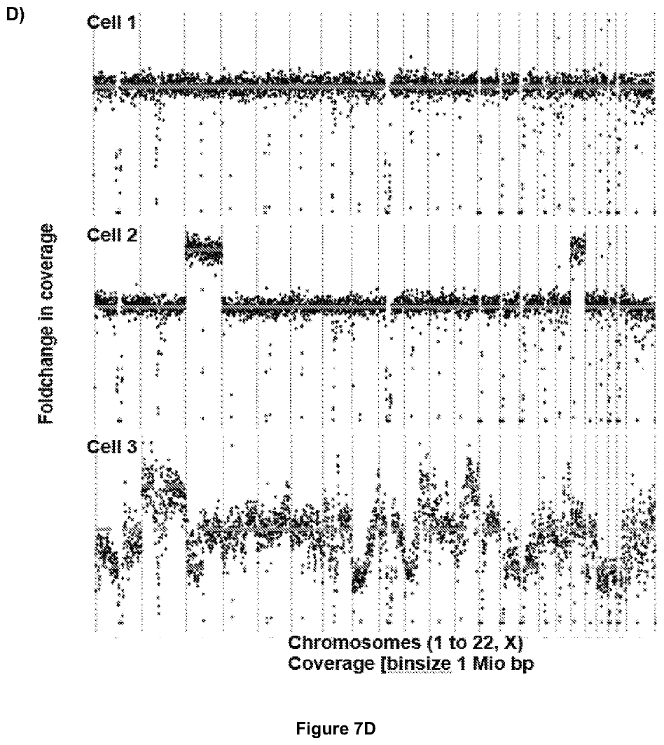

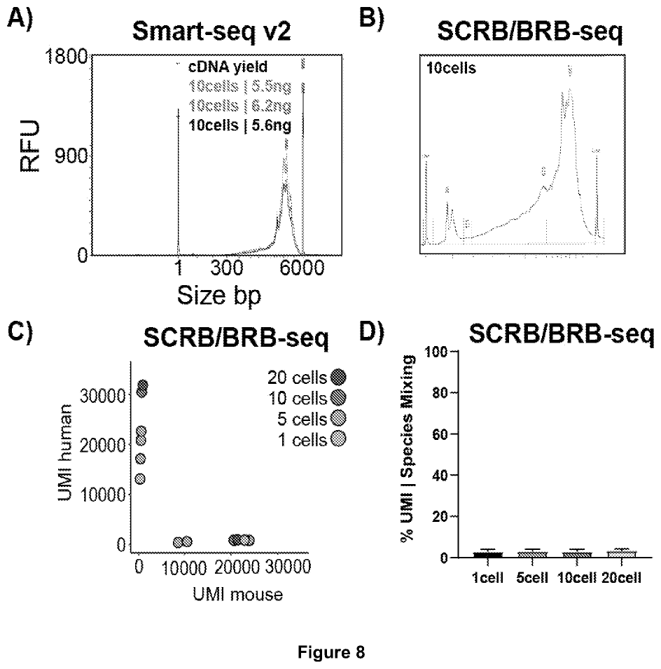

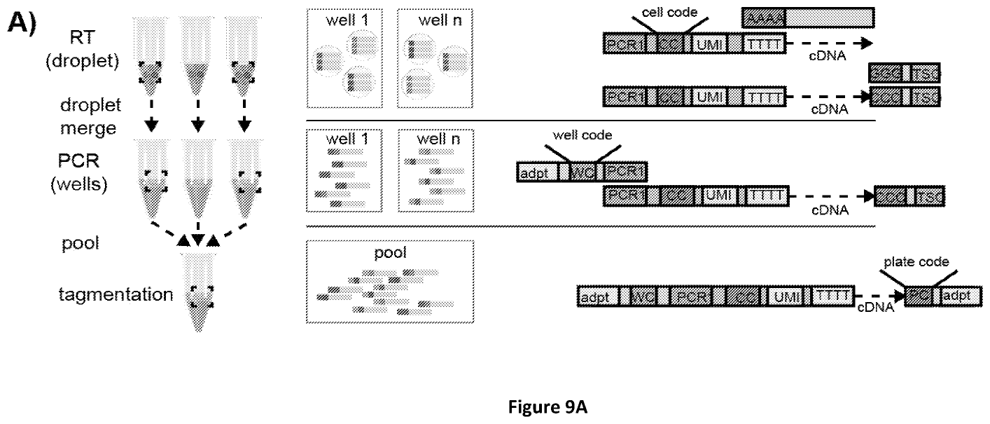

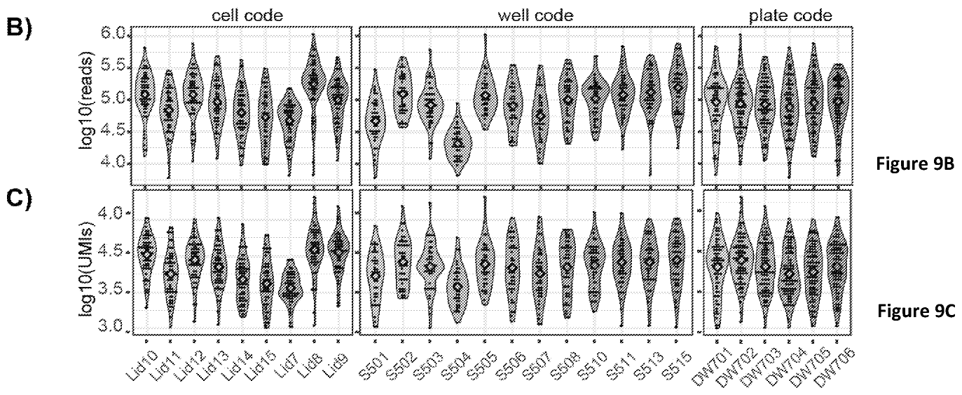

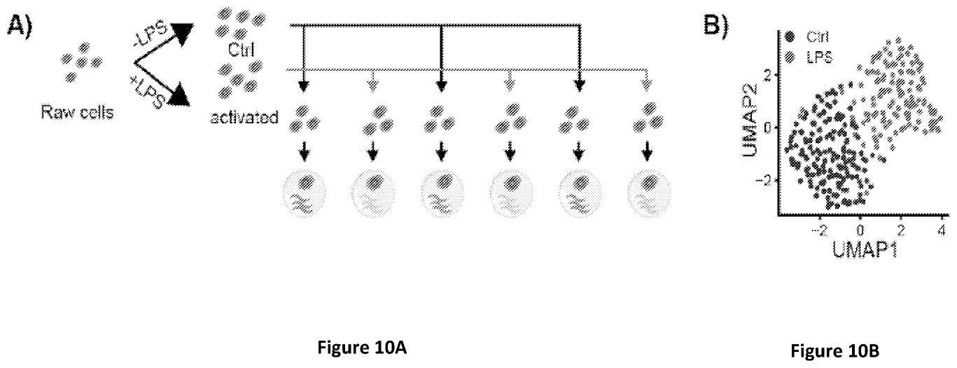

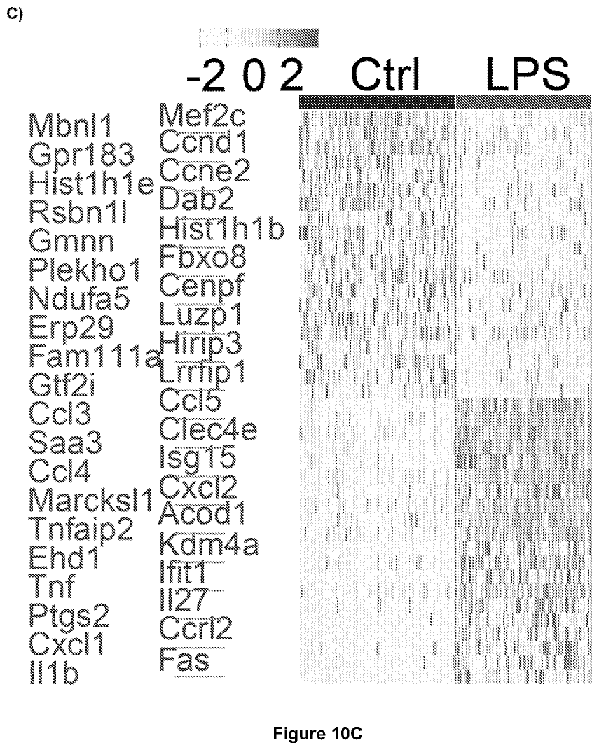

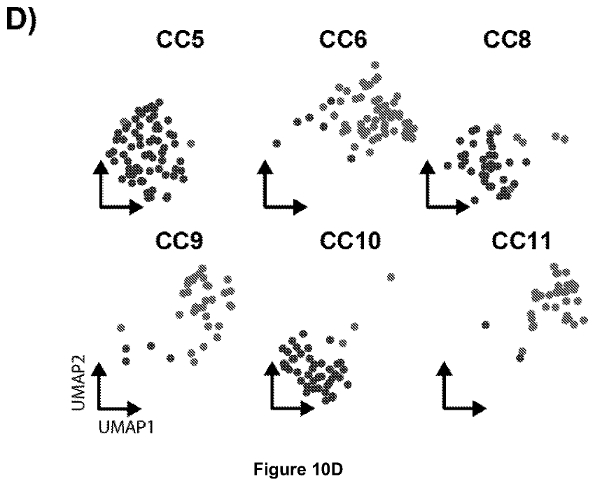

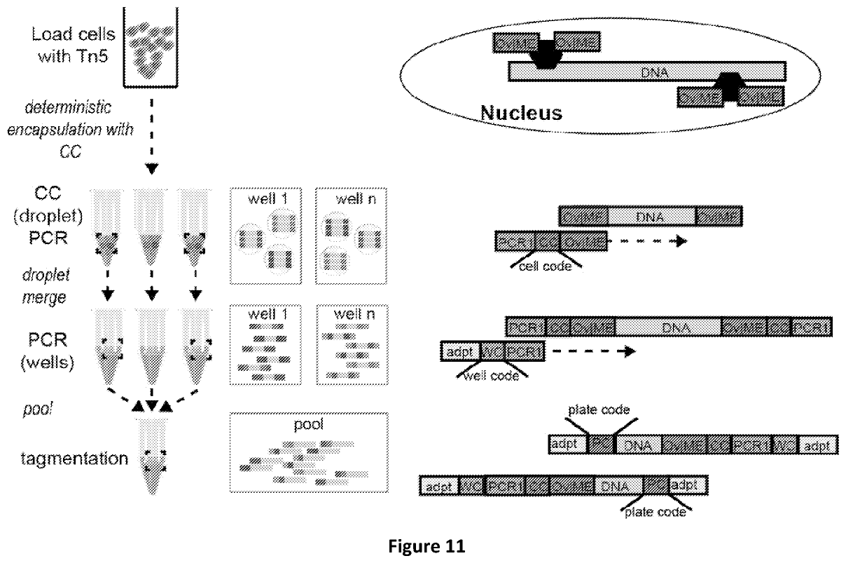

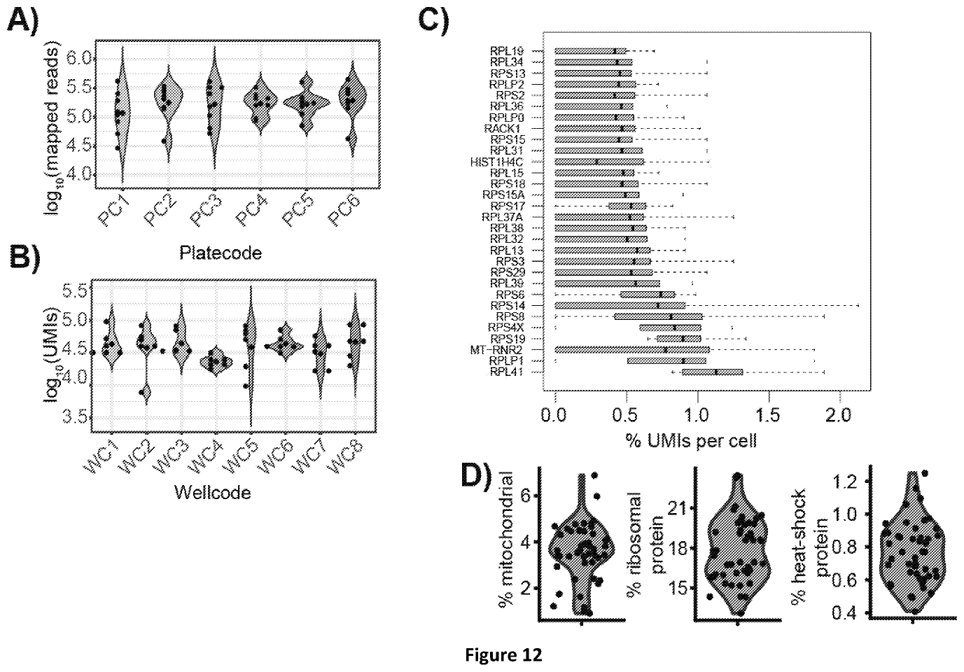

The system may comprise a device enabling quantification of entities as illustrated in FIGS. 1A and 1B. Entities are flown into the chip from a pressurized container 102, 104. Entities are imaged 101, blob detection applied, and the number of entities determined that pass through the primary counting chamber 103. The device enables dilution of the entities by incorporation adding dilution buffer through a channel connected to the sample channel 105, 106, 107, subsequent to quantification in the primary counting chamber 108 and prior to transfer to adjacent fluidic connectors and/or systems.

In an embodiment the device enabling quantification of entities, encompasses a secondary counting chamber 111, to which prior dilution buffer addition 105, 107, permits, quantification of the entities after dilution and prior to transfer to adjacent fluidic connectors and/or systems 108.

The system may comprise sample supplying means 200 comprising at least one sample reservoir containing or configured to contain at least one object in a sample buffer or in a placement buffer 200, 201, 202, 203, 204, 205, 206, 207, 208, 209.

The system may comprise buffer supplying means comprising at least one buffer reservoir or buffer reservoir containing or configured to contain a placement buffer or a primary reaction buffer 200, 201, 202, 203, 204, 205, 206, 207, 208, 209.

The placement buffer may comprise components to instigate or visualize a reaction in the cellular entity or cell derived entity prior to encapsulation.

The placement buffer may be a mediator of reaction instigation and may comprise of chemical compounds or biological agents, related to the ability of response of the cellular entity.

These components may comprise soluble factors such as pattern recognition signaling instigating molecules (e.g. LPS), cytokines, chemokines and/or toxins. These components may also comprise cellular or sub-cellular entities, like eukaryotic cells, bacteria and extracellular vesicles.

These activation mediators may be used to visualize changes to cellular compartments. Cellular compartments may be labelled with fluorescent probes or may be labelled with click-chemistry. Cellular compartments may be for example the endoplasmic reticulum, the Golgi and/or lysosomes. These cellular reactions may also be nuclear transcription factor translocations. Transcription factors may be genetically fused to fluorescent proteins or attached to a fluorophore via click-chemistry.

The placement buffer may be mediator of visualizing cellular reactions and may comprise reagents that respond to intracellular changes in the biochemical composition of the cell, such as oxygen content, metabolites and/or ions.

The placement buffer may contain for example, Fura-2, Fura Red, Indo-1, CoroNA Green, SBFI, Mag-Fura-2, Magnesium Green, FluoZin-1 and/or FluoZin-3. For pH assessment the placement buffer may also contain for example pHrodo Green or pHrodo Red. For membrane potential asessemtne the placement buffer may contain FluoVold, Bis-(1,3-Dibutylbarbituric Acid)Trimethine Oxonol (DiBAC4 (3) and/or di-3-ANEPPDHQ.

The placement buffer may also function to aid illumination-based activation of cell processes such as rodhopsins. Illumination-based activation may also be used to instigate re-folding of photo-convertible proteins.

The primary reaction buffer may be configured to perform a first reaction, the reaction being for example an enzymatic or chemical reaction.

The reaction performed by the primary reaction buffer may be a reverse transcription reaction, a polymerase chain reaction, DNA bisulfite reaction, labelling nascent mRNA reaction, click-chemistry DNA reaction, click-chemistry protein reaction, transposition of accessible chromatin, an antibody-based labelling reaction, a transposition of DNA reaction or any combination thereof.

The system comprises at least one microfluidic chip or microfluidic device 408.

Microfluidic refers to the manipulation, control or behavior of fluids that are geometrically constrained to a small scale such as sub-millimeter at which surface forces dominate volumetric forces. The size of channels, chambers may be comprised between 100 nanometers to 500 micrometers, preferably 5 to 200 micrometers.

The microfluidic device comprises a first imaging chamber 319. The microfluidic device comprises a first microfluidic channel 318a for transporting the at least one object from the sample supplying means 200 to the first imaging chamber 319.

The first imaging chamber 319 may comprise a hollow space, for example square, rectangular, or spherical, comprising one or more walls delimiting the hollow space and in which the object may be transported from the first microfluidic channel 318a.

The first imaging chamber may comprise a fluidic entry connected to the first microfluidic channel 318a and a fluidic exit from which the at least one object may exit the first imaging chamber.

The first imaging chamber may be of a size comprised between 10 and 500 micrometers.

The first imaging chamber 319 may be partially or completely transparent so that the inside of the hollow space and subsequently the object that may be inside the first imaging chamber is observable by detection means, and/or phenotypical assessing means 101.

The microfluidic device further comprises an encapsulation area or structure 320 in which the at least one object is encapsulated or co-encapsulated with a quantity or a definable quantity or a pre definable quantity of the primary reaction buffer in a droplet. The quantity of the primary buffer encapsulated with the at least one object may be definable, controlled and precise.

The encapsulation area or structure 320 may be arranged in the first imagining chamber 319 or after the first imaging chamber in fluidic connection with the first imaging chamber, for example with the fluidic exit.

The encapsulation area or structure may comprise channels in a T-junction or Y-junction configuration, in a co-flow configuration, in a flow-focusing configuration. Channel geometries may contain height variations to facilitate step emulsification and thereby encapsulation of the at least on particle.

The microfluidic device comprises a second microfluidic channel 318b for transporting the reaction buffer 302 from the buffer supplying 200 means to the microfluidic chip or to the first imaging chamber 319 or to the secondary imaging site 321 or the encapsulation area 320.

The second microfluidic channel 318b may also transport the placement from the buffer supplying means 317 to the microfluidic chip or to the first imaging chamber 319 or to the secondary imaging site 321 or the encapsulation area 320.

Several fluidic entries may be arranged on the first imaging chamber 319 to connect the chamber with microfluidic channels such as the second microfluidic channel 318b, 317, 302.

The microfluidic device may comprise a secondary microfluidic inlet to provide the primary reaction buffer 320.

The secondary microfluidic inlet may be placed prior to the placement buffer inlet, subsequent to the placement buffer inlet, subsequent to the primary imaging chamber 319, prior to the secondary imaging chamber 320 or subsequent to secondary imaging chamber 321.

The microfluidic device comprises an oil inlet 307 for introducing an oil that supports droplet formation into the microfluidic chip or a droplet forming substance inlet for introducing a droplet forming substance into the microfluidic chip 408.

The oil inlet or droplet forming substance inlet is in fluidic connection with the microfluidic chip 408 at the encapsulation area 320.

The ratio between the sample buffer containing the at least one object and the placement buffer may be, for example, ranging from 100:1, 10:1 4:1, 2:1, 1:1, 1:2, 1:4, 1:6, 1:10 or 1:100.

The ratio between the sample buffer containing the at least one object and the primary reaction buffer may be, for example, ranging from 100:1, 10:1 4:1, 2:1, 1:1, 1:2, 1:4, 1:6, 1:10 or 1:100.

The ratio between the sample buffer containing the at least one object and the oil providing inlet may be, for example, ranging from 100:1, 10:1 4:1, 2:1, 1:1, 1:2, 1:4, 1:6, 1:10 or 1:100.

The term encapsulation or co-encapsulation refers to the encapsulation of one or several particles such as a cell, a cell derived entity, a cellular entity, a cellular compartment, a bacteria, a microsphere, a protozoan, an algae, a spore, a molecule, an enzyme, a microparticle, a vesicle, a microvesicle or a microorganism.

Cellular entities may comprise peripheral blood mononuclear cells, circulating tumor cells, patient-derived leukemic cells, patient-derived lymphoma cells, patient-derived auto-immunogenic cells, cells derived from organoids, cells derived from tissues or cells derived from cell cultures.

The microfluidic device 408 may comprise an oil supporting droplet formation microchannel 307 or droplet forming substance microchannel connected to the encapsulation area 320 to place the at least one object, contained in the sample buffer and/or the placement buffer together with the primary reaction buffer in direct contact with the oil that supports droplet formation or the droplet forming substance.

The microfluidic device 408 enables to extrude a defined amount of liquid containing the sample buffer and/or placement buffer, with a definable amount of the primary reaction buffer together with the at least one object into the encapsulation area 320 via pressure appliance 316 to the placement buffer 317 and/or primary reaction buffer inlet 302. The extruded liquid is then sheared into a single droplet of defined volume, by applying pressure to the droplet-supporting oil inlet 307.

The microfluidic device comprises a droplet microchannel or tubing or capillary for transporting the droplet, in fluidic connection with the encapsulation area or the oil supporting droplet formation microchannel 307 or droplet forming substance microchannel, termed droplet evacuation means.

The encapsulation area 320 and the droplet means may be separated by a Quake-valve. The, for example Quake-valve, may be used to sort droplets containing an entity of choice 305.

The microfluidic device or microfluidic chip may further comprise a secondary oil inlet 307, 308 separated from the droplet formation microchannel via at least one valve, for example a Quake-valve or a T-valve, enabling to increase the speed of ejection of the formed droplet from the microfluidic chip by 10-fold. The secondary oil inlet is in fluidic connection subsequent to the droplet sorting means and connected to the microfluidic droplet exit means.

The system may also comprise a detection means 101 configured to detect the passage of the at least one object through the first imaging chamber.

The system or the microfluidic chip may comprise at least one primary valve configured to stop the flow of the sample buffer comprising the at least one object when the detection means detect the passage of the at least one object through the first imaging chamber. The at least one valve may be a microfluidic valve, which may be Quake-Style valve or a T-valve. Preferentially, the at least one valve is arranged on the first microfluidic channel 318a.

The system may further comprise at least one secondary valve configured to enable flow-driven over-pressure of same valve for supplying primary reaction and/or primary sample buffer 302.

The at least one secondary valve may also be configured to enable flow-driven over-pressure of same valve for supplying placement buffer 317.

The secondary valve may be configured to stop the flow of the primary reaction and/or primary sample and/or placement buffer, as to relocate the entity detected in the primary imaging chamber 319 to the secondary imaging chamber 321 via a microfluidic channel.

The detection means 101 detect the passage of the at least one object through the second imaging chamber 321.

The at least one secondary valve may be a microfluidic valve, which may be Quake-Style valve.

The system may further comprise at least one tertiary valve configured to enable flow-driven over-pressure of same valve for supplying primary reaction buffer. The at least one tertiary valve may be a microfluidic valve, which may be Quake-Style valve.

The system may further comprise a quaternary valve configured to enable flow-driven over-pressure of same valve for shearing the extruded liquid at the encapsulation area 320.

The system may further comprise a quinary valve configured to enable the expulsion of any buffer, oil and/or entity into a microfluidic exit, by enabling to stop and permit pressure-driven flow. The quinary valve provides the means to expulse unwanted entities and liquids 304.

The system may further comprise a senary valve configured to stop or permit the pressure-driven flow of droplet-enabling oil containing the droplet containing the entity together with any buffer provided by the primary, secondary or tertiary microfluidic inlet 305. The senary valve or sorting valve provides the means to sort droplets.

The system may comprise any of the valves as solenoid valves and/or valves configured for flow-driven over-pressure.

The system may comprise any of the combinations of the configured to be actuated by one pressure inlet.

The system comprises phenotypical assessing means 101 configured to assess the phenotype of the at least one object when the flow of the sample buffer is stopped by the at least one valve, being for example the primary, secondary or tertiary valve.

The phenotype of the at least one object is assessed by the phenotypical assessing means 101 when the at least one object is at an object stopping site 319a, 321a, arranged inside the microfluidic chip or inside the first imaging chamber 319 or the secondary imaging chamber 321.

The system comprises a droplet deposition means configured to deposit the droplet comprising the encapsulated object with the sample buffer and/or the placement buffer and/or the primary reaction buffer in a well or in a well of a multi-well plate and comprising an outlet capillary connected to the droplet microchannel or tubing 211, 210, 212, 213, 214.

The multi-well plate or the microwell plate may be a flat plate with multiple wells used as small test tubes. A microplate typically has 6, 12, 24, 48, 96, 384 or 1536 sample wells arranged in a 2:3, 3:4, 4:6, 6:8, 8:12, 16:24, 32:48 rectangular matrix.

The detection means, and the phenotypical assessing means 101 may comprise a microscope or a large field-of-view microscope vertically or horizontally aligned with the first imaging and/or second imaging chamber 319, 321.

The microscope may comprise a dual-camera objective imaging and detection system or a a dual-camera single objective imaging and detection system 401, 402, 403, 404, 405, 406, 407, 408, 409, 410, 411, 412, 413, 414, 415, 416, 417, 418, 419, 420, 421, 422, 423, 424, 425, 426, 427, 428.

A tunable lens or an electrical tunable 416 lens may be integrated to adjust the focal plane of the first 319 and second imaging chamber 421, or the microfluidic chip 408.

A piezo-element may be integrated with the objective or the microfluidic chip 408 to adjust the focal plane of the first and/or second imaging chamber 419, 421.

The detection means, and the phenotypical assessing means 101 may comprise at least one laser excitation diode 422, 423, 424, 421, 427, 426, 425 or a white laser to allow epi-fluorescence imaging.

In an embodiment, the microfluidic chip comprises a second imaging chamber 321 connected or in fluidic connection to the first imaging chamber 319, the second imaging chamber 321 comprising the object stopping site 319a, 321a, where the phenotype of at least one object is assessed by the phenotypical assessing means 101.

The detection means 101 detect the passage of the at least one object by blob detection or fluorescence emission, machine-learning based classifier, dark-field microscopy, event-based and/or velocity-based means.

The phenotypical assessing means 101 may assess the phenotype of the at least one object by optically assessing the phenotype.

The phenotypical assessing means 101 may assess the phenotype of the at least one object by microscopy or fluorescence intensity when the at least one object passes through or stops in the first imaging chamber or second imaging chamber or the microfluidic chip.

Phenotypical assessment of the at least one object may be performed by imaging fluorescently labelled features of the object, light-sheet microscopy, optical density tomography, confocal microscopy, quantitative phase contrast microscopy or super-resolution microscopy where obtained same images are assessed for image-contained features, and/or utilized to perform live and/or implementation of prior trained machine-learning based classifiers.

The detection means 101, applied to the at least one object, may be performed based on image-subtraction of at least two images one with the at least one entity in the microfluidic device, observed with the detection means. Same images may be subjected to image manipulations as for example Gaussian blur, hole-filling, and/or binarization to identify the entity.

The at least one entity may be quantified in regard to area, circularity and/or number of centroids. The number of entities may be quantified based on average area, number of centroids and/or shape.

The detection means 101, may also employ velocity-based detection using for example multiple images of the passing at least one entity, and/or velocity-based cameras.

The detection means 101, may be coupled to machine-learning based classifiers that identify defined types of entities such as single entities and/or multiple dissociated entities and or multiple associated entities

Machine-learning based classifiers may be obtained prior to detection or during an ongoing set of detection.

The quantification and/or analysis of the detection means may be used to decide whether the entity may be imaged at high-resolution and/or encapsulated in a droplet and/or sorted for entities of choice.

Imaging may be performed for both detection and high-resolution images at varying focal depths.

Optimal focal depth may be determined automatically and/or manually via image analysis for each imaged entity. Analytical and/or automated optimal focal depth may be determined using variance of Laplacian, gray-scale variance and/or Shannon entropy. Optical focal depth may be used to adjust the microscopy and or microfluidic setup for a defined operational run and/or for obtaining images solely in the neighboring focal depths.

The system may provide high-resolution imaging of entities. Images may be analysed prior to encapsulation. Analysis of images may be used to determine, whether the entity is encapsulated in a droplet and sorted. Decision of whether the entity is processed for encapsulation depends on at least one quantifiable feature and/or prior defined machine-learning classifiers. The high-resolution images may be for example obtained using brightfield or epi-fluorescence. Epi-fluorescent quantification may be used to quantify cellular features such as for example nuclei, mitochondria, intracellular bacteria and/or lysosomes. Features may be identified using segmentation, watershed transform, Otsu's methods and/or machine-learning classifier approaches such as “Cellpose” and “Stardist”. Identified features may quantified based on manual thresholds, dotplot-like assessment and gating and/or histogram intensities and gating.

The analysis of the high-resolution images may rely on machine-learning based classifiers may be obtained prior to image analysis or during active utilization of the system.

Machine-learning classification may utilize brightfield and/or epi-fluorescence images. Machine-learning classification may utilize deep and or shallow convolutional neural networks, convolutional autoencoders for feature extraction and/or Random forest classification.

The placement buffer or primary reaction buffer 209 may comprise a phenotype barcode identifier or an entity molecular barcode or a first molecular barcode such as a barcoded oligo-nucleotides.

The sample buffer, placement buffer or primary reaction buffer 209 may comprise a culture medium, such as a bacteria culture medium or a cell culture medium.

The sample buffer, placement buffer or primary reaction buffer 209 may comprise growth matrices and/or hydrogels.

The placement buffer or the primary reaction 209 may also comprise an enzyme or a biochemistry of choice and the necessary molecules to ensure that the enzyme or the biochemistry of choice is functional.

The enzyme may be a transposase or hyperactive transposase being a transposase comprising one or several mutations increasing its enzymatic activity.

The enzyme may be a reverse transcriptase generating complementary DNA (cDNA) from a RNA template.

In another embodiment, the sample supplying means comprise several reservoirs containing at least one object, at least one cell or cellular entity dispersed in sample buffers. The different reservoirs may comprise at least one object, cells or cellular entities from different sources or samples to be sorted, barcoded and encapsulated by the system.

The sample may comprise at least one object, at least one cell or cell-derived entities dispersed in a sample buffer or placement buffer.

In another embodiment, the placement buffer and/or primary reaction buffer supplying means 200 comprise several reservoirs, containing several primary reaction buffers. The different reactions buffers may be identical or different and contain different enzyme of choice or composition of choice and same or different phenotype barcode identifiers or molecular barcode identifiers.

Phenotype or molecular barcode identifiers enable to label all molecules per cells, for example all the transcripts of a cell.

An antibody may also be used as molecular barcode identifier, the identifier being attached to the antibody.

In an embodiment, the sample buffers, placement buffers and the primary reaction buffers supplying means 200 are reservoirs of volumes ranging from 0.001, 0.01, 0.10, 1.00, 5.00, 20.00, 100.00, 1000,00 or 10000.00 ml.

The reservoirs may be pressurized to enable flow from the reservoir into the microfluidic device via a capillary connecting the reservoir 201, 202, 203, 204, 205, 206, 207, 208, 209 to the microfluidic device 408.

In an embodiment, the sample buffer, the placement buffer and/or the primary reaction buffer supplying means 200 comprises comprise syringes in volumes ranging from 0.01, 0.05, 0.10, 0.25, 0.50, 1.00, 5.00 and 50.00 ml. Syringes are connected via capillaries with inner diameters ranging from 0.025, 0.100, 0.250, 0.500 and 2.000 mm, to the microfluidic device. Syringes are pressurized to enable flow in conjunction with the actuation of any of microfluidic valves.

In an embodiment, the sample buffers, placement buffers and the primary reaction buffers supplying means 200 comprises a circular 205 and/or XY stage structure that can be rotated 204 and/or moved linearly by an actuator.

In an embodiment, the capillaries 202 connecting the buffer reservoirs, are interconnected subsequent to the reservoir and prior to the connection to the microfluidic device 408.

In an embodiment, each reservoir may be pressurized individually.

In an embodiment, any combination of sample buffers, placement buffers and/or primary reaction buffers supplying means 200 may be pressurized by the same pressure source.

In an embodiment, the sample buffer, the placement buffer and/or the primary reaction buffer supplying means 200 comprises a rotary manifold. The rotary manifold employs a defined number of capillary inlets connected to different reservoirs containing any one of the buffers. The reservoirs are pressurized. The number of capillary inlets on the rotary valve may range from 2, 4, 6, 12 to 48 inlets. The rotary manifold is equipped with a central cylinder containing hollow pipe-like structures of 0.05 to 2.0 mm inner diameter. The cylinder can be rotated in a manner that the pipe like structures align with a defined number of the buffer inlets connected to the reservoirs. Thereto, enabling the flow of a defined reservoir into the at least one capillary connected to the central cylinder's exit. Such a rotary manifold is commercially available by Advanced MicroFluidics SA.

The system may contain off-chip valves placed prior to the inlet of the microfluidic channel 308. For example subsequent to the sample buffers, placement buffers or primary reaction buffer reservoirs 200.

The sample buffers, placement buffers and primary reaction buffers supplying means/reservoirs 200 are placed in close proximity to the microfluidic device, so as to reduce dead volumes in the capillaries 202 connecting the reservoirs to the respective microfluidic inlet. The distance between the reservoir and the microfluidic inlet defines the capillary length and ranges from 1, 5, 10, 20, 50 and 100 cm.

In an embodiment, any combination of sample buffers, placement buffers and/or primary reaction buffers 209 may be stored in designated reservoirs in a circular structure 205. The circular structure may comprise a plurality of reservoirs containing at least one object, objects, cells or cellular entities, primary buffers or primary reaction buffers. Samples, primary buffers or primary reaction buffers being contained in different reservoirs may be arranged in the same circular structure 205.

The sample supplying means, the primary buffer and the primary reaction buffer supplying means 200 may further comprise an inlet capillary connected to the microfluidic chip and configured to collect the at least one object, the cells or cellular entities and primary buffers from the reservoirs and introduce the at least one object, the cells or cellular entities or primary buffers in the microfluidic chip.

The inlet capillary may face the top of the circular structure 205 where sample and primary buffer reservoirs are arranged. The inlet capillary may be in a fixed position.

Movement of the circular structure containing the reservoirs is carried out parallel to its axis of rotation 204, 205, 201 from a first position in which the inlet capillary 202 is located outside a reservoir to a second position in which the inlet capillary is inserted into a reservoir.

The circular structure may be rotated to change the reservoir facing the inlet capillary 202 and to enable a reservoir of interest to face the inlet capillary enabling the content of the reservoir to be introduced in the system of the microfluidic chip 408.

The displacing means may comprise at least one linear rail 204 and at least one pneumatic cylinder 201 and/or a motor to displace the circular structure 205 toward the inlet capillary 202.

The inlet capillary 202 may further comprise sealing means 208 configured to seal the reservoir 209 in which the inlet capillary 209 is inserted. The sealing means may comprise a gasket or seal, such as a silicon or rubber seal 208.

The reservoir containing the inlet capillary may also comprise pressurization means 207 such as an additional capillary connected to a pressure source, such as a pump, configured to pressurize the reservoir 209, 203 in which the inlet capillary 202 is inserted to drive the at least one object, the cells or cellular entities, sample buffers, placement buffers and/or primary reaction buffers in the inlet capillary and in the microfluidic chip 408.

The droplet deposition means 210, 211, 212, 213, 214, 215 is configured to deposit the droplet in a single well or in one or several of the wells of a multi-well plate 213.

In an embodiment, droplets are deposited in a capillary 212. The capillary may be transparent.

In an embodiment, the droplet deposition means 210, 211, 212, 214 is carried out by moving the capillary 212 connected to the droplet exit of the microfluidic device 408, in XYZ position so as to eject the droplet in a single well or in one or several of the wells of a multi-well plate 213.

The droplet deposition means 210, 211, 212, 214 may comprise plate displacing means configured to displace horizontally and/or vertically the multi-well plate 213 relative to the outlet capillary 212.

In an embodiment, the droplet deposition means comprises a multi-well plate holder that can be moved in XYZ direction 215. The outlet capillary 212 is in a fixed position. The outlet point of the outlet capillary is fixed and the well or single-well of a multi-well 213 is moved towards the outlet point of the outlet capillary to capture the droplet in the designated well.

The plate displacing means may comprise at least one linear rail or a plurality of rails, for example two or four rails, to move the plate horizontally in the X or Y directions 215.

In an embodiment, the droplet deposition means may be an outlet capillary 212 connected to an actuator configured to displace the outlet capillary from a first position in which the outlet capillary is located outside a well of the multi-well plate facing the outlet capillary to a second position in which the outlet capillary is located inside a well of the multi-well plate 211.

The outlet capillary 212 may further comprise sealing means configured to seal the reservoir in which the capillary is inserted.

In an embodiment the outlet capillary 212 may be able to puncture a sealed plate for droplet deposition.

In an embodiment, several outlet capillaries 212 are connected to a rotary manifold 205, where the central inlet of the rotary manifold may be the droplet deposition capillary connected to the microfluidic device 408. Therefore, all non-centered outlets of the rotary manifold can be loaded with a droplet and the droplets expulsed from the at least two outlets into different wells of a multi-well plate.

The microfluidic chip 408 may further comprise a discarding outlet with a discarding valve configured to open for discarding an unwanted object or droplet comprising the unwanted object 304. The process of discarding may be carried out by the quinary valve.

For evaporation protection of the deposited droplets the well or the plurality of wells may be pre-loaded with components in liquid state to provide either evaporation protection and/or compensatory evaporation means.

In an embodiment, the well or the plurality of wells 213 may be pre-loaded with stuffer droplets comprising the placement buffer and/or the primary reaction buffer.

The stuffer droplets may be generated by co-flowing droplet-aiding oil and a given buffer on a microfluidic device.

Stuffer droplets may range in volume from 0.5, 1.0, 2.0, 5.0, 10.0, to 100.0nl.

Each well may be loaded with stuffer droplets ranging in number from 50, 100, 500, 1000, 10000 to 100000 stuffer droplets.

The well or the plurality of wells 213 may comprise stuffer droplets with a well barcode identifier, such as a second molecular barcode.

The well or the plurality of wells 213 may comprise stuffer droplets with a plate molecular barcode, such as a tertiary molecular barcode.

In an embodiment, the stuffer droplets may comprise a secondary reaction buffer to perform a secondary reaction inside the well of the plurality of wells, the stuffer droplets being configured to be merged with the droplet or the droplet consortia comprising the at least one object and the primary reaction buffer.

The secondary reaction buffer may be configured to perform a secondary reaction, the reaction being for example an enzymatic or chemical reaction.

Reaction performed by the secondary reaction buffer may be enzyme-based DNA transposition and/or polymerase chain reactions and/or click chemistry reactions and/or protease-based digestion reaction,

The well or the plurality of wells 213 may comprise a well barcode identifier, such as a second molecular barcode.

The well barcode identifier may be loaded in the well directly or in the stuffer droplets.

Each well may contain different stuffer droplets comprising the primary reaction buffer, for example without the enzyme or an additional buffer to perform an additional biochemical or chemical reaction.

Once the droplet encompassing at least one object, at least one cell or cellular entity and the primary reaction buffer is deposited into the well, the droplet and the stuffer droplet or a plurality of stuffer droplets may be merged via electrostatic forces before or subsequent to an initial reaction.

The electrostatic force may be established by an electric field of the supplied in the range of 240, 1000, 5000, 10000, 50000, 100000 Volts.

The electrostatic forces may be applied for example by two metal plates above and below a well or multi-well plate 213.

The electrostatic forces may be applied for example by two metal plates besides 602 a well or the rows of multi-well plate 213.

The system may also comprise a calculation means or a processor, for example an embedded computational system and/or conventional computer connected to detection and phenotypical assessing means and configured to capture or obtain data for detection and phenotype data of at least one object from the detection and phenotypical assessing means and link the detection, phenotype data with the phenotype barcode identifier encapsulated with the at least one object and the multi-well location based well identifier in which the droplet is deposited.

The calculation means or the processor may also be connected to the other elements of the system and configured to control and command these elements to permit deterministic microfluidic operation of the system.

The system may include a memory (for example, semiconductor memory, HDD, or flash memory) configured to store or storing at least one program or processor executable instructions. The at least one program or processor executable instructions may comprise instructions permitting, for example, to control and command the sample supplying means, the buffer supplying means, the pressure source, the detection means, the phenotypical assessing means, the droplet deposition means, the valves arranged in the system, and the other system elements. The processor executable instructions may comprise instructions permitting to obtain/receive and process the captured image or image data from the detection means and/or the phenotypical assessing means.

The calculation means or a processor and the memory can be, for example, included in a computer, portable laptop or a portable device such as a smart phone or device. The program or processor executable instructions can be provided, for example, as custom Matlab functions, Phyton, C++ and/or VHDL.

The processor executable instructions can include instructions permitting various different actions concerning capturing and processing images and image data of the present disclosure.

The processor executable instructions are provided or obtained by the processer for execution.

The processor may capture or obtain and digitally store in a dedicated memory the detection and phenotype data such as image and/or video of the at least one object or fluorescence profiles.

The processor may be further configured to operate droplet deposition means to deposit the droplet in a specific well depending on the phenotype of the encapsulated object.

The processor may be configured to operate the detection means and the phenotypical assessing means.

The processor may be further configured to detect movement of the at least one object, using the captured images from the detection means.

The processor may be configured to determine from the images when the at least one object reach a predetermined stop position, and further configured to trigger the closure of a primary, secondary or tertiary valve depending on through which channel the at least one object is arriving.

The processor may be configured to control, trigger the closure or the opening of the primary, secondary, tertiary, quaternary, quinary and/or senary valve.

The processor may be configured to optically inspect the at least one object in the first and/or second imaging chamber at a predetermined stop position, and to proceed with encapsulation or to flush out the at least one object to the discarding outlet.

In an embodiment, optical inspection is manual human-based allowing to perform entity by entity decisions based on the images provided and human decision to decide for encapsulation or discarding.

In an embodiment, optimal inspection and decision for encapsulation is performed by a computer executed program based on pre-defined parameters such as for example entity circularity and size. This for example allows for the sorting or exclusion of cellular doublets.

In an embodiment, optimal inspection and decision for encapsulation is performed by a computer executed program based on pre-defined parameters, such as fluorescence presence, fluorescence intensity, the identification of a plurality or defined combination of fluorescence signals or absence of a fluorescence signal.

In an embodiment, the fluorescence quantification may be performed by avalanche diodes quantifying photons in the emission/imaging light path.

In an embodiment, the fluorescence quantification may be performed by photo multiplier tubes quantifying photons in the emission/imaging light path.

In an embodiment, optimal inspection and decision for encapsulation is performed by a computer executed program based on entity features, that relate to quantifiable features in the imaged entity. These may be for example organelles, such as mitochondria, Golgi apparatus, endoplasmic reticulum, lysosomes, bacteria and/or the nucleus.

In an embodiment, entity features are quantified using fluorescently label organelles such as for example organelles, such as mitochondria, Golgi apparatus, endoplasmic reticulum, lysosomes, bacteria and/or the nucleus. Quantification of same features may be carried out using fluorescence intensity per entity, number of features, size of features or any combination thereof.

In an embodiment, the entity features are assessed using a machine-learning derived classifier. The classifier may be obtained via deep-learning approaches such as inception-derived networks or convolutional autoencoders pre-pended to neural network-based classifications.

The processor may be configured to close the discarding quinary valve and open the sorting senary valve if encapsulation is chosen.

The system may further comprise a collection channel and an outlet and the collection senary valve configured to open only for collection of encapsulated objects; and/or waste channel and outlet and the quinary waste valve configured to open to flush out an unwanted object.

The present disclosure also concerns a method of operating the system for the phenotypical profiling of at least one object and the deterministic nanoliter-droplet encapsulation.

The method may be a computer executed or computer implemented method.

The method may comprise the step of providing a system for the phenotypical profiling of at least one object and the deterministic nanoliter-droplet encapsulation according to the present disclosure.

The method comprises the step of introducing at least one object from the sample supplying means into the first imaging chamber through the first microfluidic channel 318a.

The method may comprise the step of stopping the flow of the sample buffer containing the at least one object when the detection means detect the passage of at least one object through the first and/or second imaging chamber.

The method may further comprise the step of assessing the phenotype of the at least one object when the flow of the sample buffer and/or placement buffer is stopped.

The method may comprise the step of introducing the primary reaction buffer from the buffer supplying means into the microfluidic chip through the microfluidic channel.

The method may comprise the step of introducing the oil that supports droplet formation into the microfluidic chip or the droplet forming substance into the microfluidic chip through the oil inlet or the droplet forming substance inlet.

The method may comprise the step of imaging features instigated by the placement buffer prior to encapsulation.

The method may further comprise the step of transporting the at least one object and the sample buffer, the placement buffer and/or the primary reaction buffer to the encapsulation area or to the structure for encapsulation by the droplet.

The method may comprise the step of transporting the droplet to the droplet deposition means through the droplet microchannel or tubing for deposition of the droplet in a well or in a well of a multi-well plate.

The method may comprise the step of providing a phenotype barcoding identifier in the primary or secondary reaction buffer that enables labelling the at least one object encapsulated in the droplet.

The method may further comprise the step of providing a well barcode identifier enabling to uniquely labelling the encapsulated object per well.

The well barcode identifier may be added directly in a specific well or be comprised in stuffer droplets preloading a specific well.

The present disclosure also concerns a droplet containing at least one object encapsulated with the primary reaction buffer obtained according to the present method or with the disclosed system.

Alternatively, the disclosure also concerns a plurality, or a group of defined droplets placed in one well, each containing at least one object, with each droplet containing one defined primary reaction buffer and/or phenotype or molecular barcode identifier, obtained according to the present method or with the disclosed system.

The disclosed method and system streamlines molecular profiling from the initial cell entity input to barcoded molecules ready to profile (e.g. sequencing, mass spectrometry): I) Cell quantification and phenotypical profiling II) extraction of molecular type of choice, III) molecular type quantity normalization, IV) molecular type per cell entity barcoding and V) molecular type barcoding per reaction vessel in one stream-lined process of which I) to IV) take place within nanoliter-droplets in the framework of a single-cell entity microfluidic process with integrated imaging and V) being carried out by for example appending a DNA barcode to each DNA molecule present in the well, thereby drastically reducing the experimental complexity, time and costs.

The disclosed system or technological platform also functions as a single-cell processor and enables Integrated Robotic Imaging and Single-cell RNA-sequencing, from here on referred to as IRIS. Due to its deterministic microfluidic processing, IRIS enables to process every input cellular entity from the get-go, thereby making the efficient processing of rare cellular entities possible and enables the deposition of same entities within droplets in any container, being it for example single- or multi-well containers. Importantly, the integrated imaging components of IRIS enable the acquisition of phenotypical imaging data on a per cellular entity basis and permits sorting of cells previous to encapsulation. Therefore, each cells images can be directly linked to the molecular profile on a per cell basis.

At its core, this method consists of the disclosed system or IRIS platform that enables to perform each of the following elements on at least one cell or cell-derived entity in the form of active and deterministic nanoliter-droplet handling:
1. The IRIS platform, for example, comprising:
   Microfluidics chip or system (based on the international PCT patent application no. WO2018/051242 A1, which is hereby incorporated by reference) and its descendants, encompassing peripherals for fluid flow & valve control, entity enumeration & adjustment and automated liquid biochemistry multiplexing
   Detection means and/or phenotypical assessing means comprising a microscope encompassing a dual-camera objective or single imaging and detection system, together with a tunable lens or an electrical tunable lens and laser diode excitation for epi-fluorescence imaging
   Computational processing framework coupling real-time image analysis and storage together with microfluidic control
   Evacuation capillary synchronized with an XYZ-stage for droplet deposition
2. A single particle solution of cells or cell-derived entities
3. A primary or reaction buffer, comprising or consisting, for example, of:
   Detergent for lysis
   Barcoding molecules (e.g. oligo-nucleotide)
   Buffer
   Enzyme
4. Stuffer droplets with buffers:
   Opti-prep
   Additional enzymatic reaction
   Primary reaction buffer lacking the enzyme

The method allows performing cellular entity's molecular profiling (e.g. RNA/DNA) to be barcoded in an automated procedure:

1) Using large-field of view microscopy, each cellular entity is detected via machine-vision (blob detection). Upon detection, the flow of cells on the microfluidic system is stopped here using a first valve such as a Quake-Style valves or T-valve, which block liquid flow by pressing a membrane into the flow channel (process described in detail in FIG. 3).

Subsequently, the sample inlet is over-pressured enabling continuous flow of the entity to the imaging and encapsulation point. Over-pressure is abrogated when the entity is detected, and the entity can be examined (e.g. by imaging) prior to encapsulation. Defined nanoliter volumes of entity-containing buffer with the entity and reaction buffer are co-expulsed and sheared with oil to form a droplet. The generated nanoliter-droplet containing the entity and the reaction buffer (containing one distinct barcode) are deterministically evacuated to a multi-well plate. Images per entity are digitally stored.

1a) If processing of multiple single entities (e.g. scRNA-seq), each detected entity is deposited in a new well of the multi-well plate. If several entities are to be deposited in the same well of the multi-well plate, reaction buffer containing a barcoded oligo-nucleotides is replaced within the microfluidic system.

1b) If bulk profiling of samples (e.g. RNA-seq) is desired, multiple entities with the same barcoded oligo-nucleotide are deposited in each well of the multi-well plate.

2) The first enzymatic barcoding reaction is carried out within the nanoliter-droplets evacuated from the system or IRIS within the multi-well plate. The multi-well plate is pre-loaded with a defined amount of nanoliter stuffer droplets containing the reaction buffer without the enzyme.

3) Nanoliter-droplets are merged via electrostatic forces and an additional barcode is appended on a per multi-well unit basis, via an overhang PCR utilizing an appendix of the initial barcoding procedure.

### Experimental Validation

The inventors validate our approach by a series of experiments demonstrating that:
1. Concentration of entities can be adjusted in real-time per sample.
2. Entities and primary reaction buffer mix can be automatically introduced to the microfluidic chip.
3. Entities can be positioned by over-pressuring the first valve or sample inlet valve.
4. Entities can be encapsulated with primary reaction buffers of different composition.
5. Entities in nanoliter-droplets can be automatically deposited in a multi-well format.
6. Nanoliter-droplets are stable reaction containers and can be merged using electric forces.
7. Cellular entity's nucleic acid molecules can be transformed into per-sample barcoded cDNA/DNA within the nanoliter-droplet.
8. By using different barcoded oligonucleotides in the reaction buffer, consortia of multiple cell entities can be processed per multi-well unit.

### Quantification and Automated Microfluidic Entity Concentration Adjustment

Enumeration of cellular entities is a general task prior to applying molecular profiling and/or isolation of the molecular entity. In order to enable direct, loss-less down-stream processing, entities may be enumerated. Thereby, the entity concentration can be adjusted accordingly, prior to processing on the microfluidic system, particularly in the case for automated multi-sample processing, with each of the samples containing varying concentrations of entities.

Initially, entities may be enumerated utilizing a miniaturized microscope monitoring a see-through capillary, directly subsequent to the sample port (FIG. 1A). Using machine-vision, which is implementing image subtraction to identify circular entities of a definable size, entity enumeration may take place directly after the sample port.

In order to enable adjustment of the entity concentration, the sample inlet capillary is intersected with a capillary feeding from a sample dilution buffer. At the intersection between the sample capillary and the sample dilution capillary, both solutions are mixed. The downstream microfluidic system requires identical pressure for processing.

Therefore, the sample port and the sample dilution buffer port are pressurized individually using high precision pressure regulators, and pressures are adjusted according to the entity concentration via direct and continuous feedback from the sample monitoring. As an alternative enumeration approach, entities can be quantified previous and subsequent to intersecting the dilution buffer and sample capillary, thereby enabling rapid adjustments of entity concentration and a continuous equal entity concentration, particularly necessary in the context of fluctuating experimental conditions such as sampling from cell cultures or differing sample buffer conditions (FIG. 1B).

The automatic microfluidic entity concentration adjustment will also enable dilution of, for example cell culture or entity storage buffers, which may otherwise inhibit enzymatic reactions such as reverse transcription.

By diluting the entity buffer prior to the encapsulation process, optimal conditions for the initial step of molecular profiling can be established without additional manual labor.

### Automated Entity and Reaction Buffer Supply by Precision Pressure Application

Spatial coordination of entities and on-demand droplet generation requires precise regulation of fluid flows in the microfluidic system.

To this end, the inventors developed a precision pressure source based on a piezo-bender valve (Festo SE & Co. KG) that is actuated by a high-voltage driver integrated circuit (IC).

The high-voltage is connected to a microcontroller unit (MCU) that regulates the output voltage.

An electronic pressure sensor, connected to the MCU, monitors the pressure outlet of the piezo-bender valve.

The piezo-controlled board was developed by octanis instruments.

Regulation of pressures is achieved by measuring the pressure (is-value) at the outlet of the piezo-bender valve, comparing the is-value against the intended set-value, calculating the compensation value, and transmitting this value to the high-voltage IC.

The calculation of the compensation value is achieved via a proportional (P) control algorithm, but could be as well achieved/augmented with integral (I), derivative control (D), or combined into e.g. a PID controller.

The inventors further devised peripheral systems allowing for automatizing global processes, namely the i) exchange of entity and/or reaction solutions and ii) targeted deposition of droplets into wells.

For point i), the inventors devised an automatized entity/reaction solution sampling device (FIG. 2a). Similarly, as for the deposition of droplets, a capillary is inserted and retracted from wells containing the solutions to be injected into the system.

As only a limited number of different reagents are used in the current use cases of the system, single wells in the form of microtubes are used as containers.

A rotary-based system was developed to switch between reactant solutions, but could be exchanged for an XY-stage.

Insertion of the capillary can be achieved by either actuating the capillary position or the vertical position of the rotary wheel (FIG. 2a).

As liquids are injected into the system by pressurization of the sample reservoir 209 or well/vessel, a manifold for the capillary was developed allowing for pressurization of the vessel with e.g. the pressure source described above (FIG. 2b/c).

Overall, by utilizing the high-precision pressure regulators, automated exchange of liquid biochemistries and samples can be achieved, aligned with the precision deposition of nanoliter-droplets into the designated reaction vessels.

For the deposition of droplets in wells, a mechanism was developed that controls the horizontal positioning (x/y coordinates) (FIG. 2d) as well as vertical positioning (z coordinate) (FIG. 2e/f).

Horizontal positioning was achieved with a linear stage actuated by stepper motors.

For vertical control of the capillary position a pneumatic cylinder was utilized.

Two different strategies were developed to insert and retract the capillary from a well (could be any other open reaction compartment).

The first mode of actuation moves the capillary up and down (FIG. 2e).

The second mode of actuation moves the reaction vessel (here multi-well plates) up and down (FIG. 2f).

Pneumatic control over the movement is either achieved by utilizing a 5/3-way valve combined with flow control valves that restrict airflow and prevent perturbation of the well contents.

Alternatively, the above described piezo-based pressure regulators can be used to precisely generate pressure ramps. The latter regulation mode is actively configurable and thus allows for optimal movement dynamics that maximize actuation speed while at the same time preventing perturbation of the reaction vessel's contents.

### Detection and Positioning of Entities by Over-Pressuring the Sample Inlet Valve

The inventors initially developed the microfluidic system to enable fully automated handling of droplets and their evacuation from the chip.

The inventors combined machine-vision with on-chip microvalves to control cell position, droplet generation, and droplet isolation on chip (FIG. 3A).

The process itself consisted of a 6-step microfluidic process, with three encapsulation steps (FIG. 3B) and two droplet handling steps (FIG. 3C):
i) cell detection and stopping,
ii) precision cell placement
iii) combination of the cell with the reaction buffer,
iv) encapsulation into a droplet,
v) isolation of the droplet from the encapsulation area, and
vi) evacuation of the droplet in a well.

Three elements may greatly contribute to enhance speed and precision as compared to the previous implementation:²⁴
1) instead of valve oscillation for cell placement, the first valve or cell valve is over-pressured to generate a leak flow for cell positioning, reducing placement times (FIG. 3D).
2) a placement buffer inlet channel, subsequent to the primary imaging chamber allowing for precision placement of the entity subsequent to detection at the secondary imaging chamber adjacent to the encapsulation area (FIG. 3F-G).
3) addition of a secondary sample port connected to an off-chip solenoid valve controlling the flow of pressurized oil, allowing a droplet that passed the sample valve to be flushed out subsequent to the sample valve being closed (FIG. 3A).

To test the efficiency of the stopping process prior to encapsulation, cells or nuclei were manually counted and the stopping efficiency was calculated as percent of cells stopped at the designated imaging area (encapsulation point) subsequent to the initial detection for HEK 293T cells, RAW cells, embryonic stem cells or neuronal nuclei and reached 90%, 84% 81% or 80%, respectively (FIG. 3E). The microfluidic workflow thereby enables deterministic encapsulation of cellular entities with at least >80% efficiency with a 3-5 folds increase in speed.

Furthermore, an alternative mode of droplet generation was developed based on the previously described approach of over pressurization of Quake microvalves.

Defined and fine-grained pressurization of the placement or sample buffer and/or the primary reaction buffer inlets allows precise injection of defined amounts of liquids, which subsequently can be sheared by activating oil-flow.

This provides two additional means to optimize the nanoliter-droplet manufacture, and allows excluding the utilization of the T-valve (or droplet generation valve) and the cell inlet valve (FIG. 3A-B):

1) The ability to adjust the pressures for both the sample and placement buffers and the primary reaction buffer inlet independently in a high-precision mode permits injection of nanoliter-volumes into the encapsulation area prior to shearing of the droplet, thereby reducing processing time per manufactured nanoliter-droplet by making the T-valve obsolete.

2) By utilizing the high-precision pressure regulators, over-pressurizing of each inlet valve can also be used to adjust the reagent composition, meaning the ratio of cellular entity containing sample buffer and the primary reaction buffer. To this end, the primary reaction buffer and/or cellular entity inlet are pressurized for defined times during droplet formation. The real-time machine-vision imaging permits to quantify droplet size, thereby enabling to adjust the reagent ratios, which is not possible with the T-valve only enabling 50/50 ratios.

Finally, the same mechanism can be utilized to place entities for examination/encapsulation without continuously opening and closing the cellular entity inlet valve, which is necessary to avoid entities to get stuck in the valving structure. For this, two inlets with two valves are placed in front of a microfluidic chamber or the first imaging chamber 319 (FIG. 3F).

Initially, the inlet valve containing the entity solution is opened and entities are injected into the chamber.

Once the chamber is filled, the entity valve is closed, and buffer is injected into the chamber from the 2nd channel by valve over-pressurization.

This way, all entities contained in the chamber can be placed for encapsulation by valve over-pressurization.

Avoiding open and closing cycles of the entity inlet valve has multiple advantages including: i) higher concentrations of entities can be processed thus increasing processing speed and ii) time overheads of opening and closing the valve are avoided, further increasing processing speed.

### High-Resolution Imaging of Positioned Entities Via a Dual-Camera Single Objective Microscopy

The deterministic positioning of a cellular entity at a designated stopping site enables the incorporation of sophisticated custom microscopes together with the microfluidics setup.

In order to provide a large field of view (FOV), required for detection and a small FOV for high-resolution imaging, the light paths for both detection & placement of the cell and imaging may be separated.

Therefore, the inventors implemented a microscope comprising or consisting of, for example, a 40×-objective & 0.9NA, (two cameras i) high-frame rate >400 fps for cell detection, ii) for cell imaging with large optical sensor); two illumination light sources (a) far-red LED (805 nm) for detection b) white LED for cell imaging); a multi-color excitation source with multiple laser diodes; a tunable lens or an electrically tunable lens (ETL) following a 4f-relay system as well as a beam expander, for example, a 2× beam-expander in the imaging path; and multiple dichroic mirrors and lenses (FIG. 4A).

The whole setup is integrated into the control system actuating valves and fluid flow via pressure regulators, image processing, transfer and data storage (FIG. 4B).

By that, the inventors were able to detect a single-cell at the primary FOV, position the cell and image it multi-modally at the secondary FOV across multiple focal planes enabled by the ETL (FIG. 4C).

The ETL, used for Z-direction focal stacking may be readily replaced together with the 4f relay by installing a piezo-element at the sample holder stage holding the microfluidic device (FIG. 4D).

Due to the separated light-paths and the matching magnification for detection and imaging, any light-based microscope can be integrated in the setup, such as confocal microscopy (e.g. spinning-disk), structured illumination microscopy, and light-sheet microscopy, etc..

Overall, the microscopic framework provides a system to enable rapid cellular entity detection and precise placement of said entity for imaging, or other examination strategies.

### Entities in Nanoliter-Droplets can be Automatically and Deterministically Deposited in a Multi-Well Format as Droplet Consortia

The main physical components of the IRIS setup consist of a custom horizontal microscope positioned over a multi-well plate handler, that moves the plate in both horizontal directions.

For vertical movement, i.e. insertion and retraction of the capillary from wells, a pneumatic actuation mechanism was integrated (FIG. 5A).

Alternatively, the actuation plate can be actuated pneumatically, allowing the capillary to stay in a fixed position.

Upon successful placement of a sample droplet in a well, the capillary is retracted, the plate-handler moved to the next position, and the capillary inserted in the next well (FIG. 5B).

Droplet deposition can also be achieved by moving solely the multi-well in XY direction and additionally in Z direction (not depicted).

To achieve tight synchronization of the microfluidics process with the mechanical elements, the inventors integrated the capillary-handler with the custom control system (FIG. 4C). Overall, the process allowed us to evacuate a defined number of droplets into wells of a multi-well plate reliably (FIG. 5C).

The product of this process is a group of droplets in one well, which the inventors term consortia, with fully defined and configurable composition. For instance, each droplet in one consortium can contain a single entity (cells, particles, etc.) combined with a unique reactant mixture.

Droplet consortia are a unique and novel concept which is enabled by the above-described instrumentation and the implemented processes.

### Nanoliter-Droplets are Stable Reaction Containers and can be Merged Using Electric Forces.

The nanoliter-droplets are required to be stable (not coalesce) during the initial enzymatic barcoding reaction to ensure that each of the molecules per entity is robustly and uniquely barcoded.

To ensure stability of reaction droplets, each well of the multi-well is supplied with a defined volume of nanoliter-droplets containing the reaction buffer, here termed “stuffer-droplets”.

The stuffer-droplets serve two purposes: 1) Evaporation protection, as they are less dense due to the reduced concentration of Opti-prep. 2) Physical separation of the nanoliter-droplets containing the entity.

Subsequent to the initial enzymatic reaction, the droplets require to be merged with secondary reagents, to for instance perform additional barcoding.

As the nanoliter-droplets cannot be merged using chemical merging or high-speed centrifugation the inventors devised an electrostatic de-emulsification approach that was independent of chemical perturbation, as the latter has been reported to inhibit PCR reactions and thus would require an additional cDNA purification step prior to PCR.⁷

To this end the inventors used a high voltage plate capacitor-like structure driven at 1.3 kV and 50/60 Hz, that fit an entire multi-well plate and allowed us to merge all droplets contained in a well across a multi-well plate simultaneously (FIG. 6).

Furthermore, stuffer-droplets can be used to store secondary reaction buffers, which can be merged with the nanoliter-droplets initially containing the cellular entity, subsequent to the initial reaction (e.g. cell lysis). As the nanoliter-droplets contain a very low volume, in the range of nanoliters of the initial reaction buffer, merging with the stuffer-droplets containing the secondary reaction buffer would sufficiently dilute out the primary buffer.

Stuffer-Droplets can be Utilized for Adjusting Nanoliter-Droplet Content and/or Providing Enzymes for Secondary Reactions

Stuffer-droplets serve as a separating agent for the reaction droplets and as evaporation protection. Furthermore, they can also function as containers to transmit small molecules (e.g. ions, buffer contents, nucleotides) into the reaction nanoliter-droplets via droplet-to-droplet osmosis.

The inventors confirmed the diffusion of such small molecules by performing the RT within the nanoliter-droplets together with Stuffer-droplets containing different dNTP concentrations and observed increasing cDNA yield correlating with increasing dNTP concentrations (FIG. 7A).

Hence, Stuffer-droplet based exchange of small molecules can be utilized to i) support the primary reaction within the reaction-droplet (see FIG. 7A) or ii) trigger a secondary reaction by the stuffer-droplet driven addition and exchange of critical ions, such as Mg2+, to initiate cleaving of Tn5-bound DNA.

Importantly, Stuffer-droplets can be added previous to encapsulation of cellular entities within reaction-droplets or subsequent to encapsulation.

As droplet-to-droplet exchange is mostly limited to small molecules, enzymatic reactions can be deposited within the Stuffer-droplets to be activated after electric forces-based merging (see FIG. 6).

To exemplify the functionality of Stuffer-droplets to store a secondary enzymatic reaction, the inventors performed whole genome sequencing (WGS) library preparation.

Initial cellular lysis and chromatin opening was performed using thermolabile 0.1 mg/ml Proteinase K with 0.5% w/v Triton X-100. Subsequently reaction-droplets were heated to 55 C for 10 min and then merged with Stuffer-droplets containing Tn5 loaded with A/B adapters for DNA transposition. DNA was amplified via overhand-PCR utilizing the Tn5 appended adapters and yielded DNA fragments of appropriate length distribution for sample-inputs down to one cell (FIG. 7C). Single cells profiled with the describe process for single cell whole genome sequencing contained distinct genomes provided insights into chromosome aberrations (FIG. 7D)

Cells and Nuclei can be Transformed into Per-Sample Barcoded cDNA/DNA within the Nanoliter-Droplet

Deterministic droplet assemblies with cellular entities enable primary enzymatic processing and/or barcoding of molecules in volumes two orders of magnitude smaller than standard approaches performed in multi-well assays.

To assess the possibility for cDNA synthesis in nanoliter-droplets the inventors first implemented SMART-seq v2^{25,26} and encapsulated 10 cells in 10 individual nanoliter-droplets and automatically deposited same droplets per unit in a multi-well. Subsequent to RT in nanoliter-droplets, the same droplets were merged with a PCR reaction buffer containing template switch oligo (TSO) primers and amplified the cDNA. Analysis of DNA concentration and size distribution confirmed the generation of high-quality cDNA from a maximum of ten cells per sample (FIG. 8A).

To enable the assembly and unique labeling of multiple cellular entities per well of multi-well plate, the inventors next implemented modified SCRB-seq/BRB-seq chemistry,^{23,27} using 1% w/v Triton X-100 as the lysis reagent. The inventors assembled droplet consortia of 1, 5 or 10 HEK 293T cells per barcode and found that the inventors were able to robustly generate cDNA with the expected size-distribution from cells encapsulated in nanoliter-droplets for RT (FIG. 8B).

Next, the inventors utilized a species mixing experiment (mixture of mouse and human cells) to test whether our system was capable of generating cDNA at single-cell resolution. In order to test this systematically, the inventors used a modified species mixing setup in which the inventors processed 1, 5, 10 or 20 cells of one species per barcode, ensuring that each well contained both human HEK 293T cells and mouse RAW 264.7 cells with two distinct cell barcodes. The inventors found that both species were separating independent of the amount of cells processed together with one unique barcode, with the tendency of more cells yielding higher numbers of UMIs per species (FIG. 8C). The proportion of mixed species per barcode was below <3% independent of the number of droplets processed.

This experiment showed that it is indeed possible to rapidly implement scRNA-seq biochemistries in droplets, process multiple entities per well independent of the number of nanoliter-droplets per sample and achieve single-cell resolution with our approach.

The inventors confirmed that the nanoliter-droplets remained integer during the initial enzymatic reaction and that there also was no barcode mixing during the PCRs as the extent of species mixing observed was equal or lower than competing methods (FIG. 8D).⁷⁻⁹

Encapsulation of cells or nuclei with an initial primary enzymatic reaction buffer, also enables the labelling of, for example open chromatin. To this end, cells/nuclei are encapsulated with a reaction buffer containing hyperactive Tn5 pre-loaded with S5 and S7 DNA molecules. Encapsulated cells are deposited as singly per unit of multi-well plate or a multitude enabling the assessment of open chromatin regions either on a per cell or per sample basis. By utilizing nanoliter-droplets for the Tagmentation reaction, used reagents, such as Tn5 enzyme, are reduced by two orders of magnitude, as only the cells that need to processed will receive Tn5, which is not possible with existing approaches.²⁸

### Deterministic Barcoding Enables Processing of Cell Consortia Per Well

Both the SMART-seq and SCRB-seq implementation of nanoliter-droplet processing of multiple cells yielded robustly cDNA from single or a defined multitude of cells enabling full-length transcript or 3′RNA-seq, respectively.

Although the processing enables fast and automated acquisition of defined RT input per well, downstream throughput was limited as the redundant sets of barcodes contained in each droplet consortium prevents from pooling consortia after RT, thus each consortium per well of the multi-well has to be independently processed up to and including the final sequencing library preparation.

For this reason, the inventors integrated a secondary barcoding layer during the initial PCR reaction, which the inventors termed the “well-barcode” (WC, FIG. 9A).

In detail, cells were barcoded as before during RT with a unique cell barcode. Subsequently to RT, the droplet consortia in each well were merged, and PCR reaction mix added to the wells. The PCR primers annealing to cDNA molecules carried the additional WC (FIG. 9A). For each well, a unique well-barcode primer was added, generating a unique well- and cell-barcode (CC) combination for each cell.

Hence, the inventors were able to pool droplet consortia early during library preparation, before any handling-intensive steps occurred, such as DNA purifications or tagmentation.

For multiplexing of multiple experiments, the i7 index, here termed plate code (PC), was utilized, appended after tagmentation via overhang PCR (FIG. 9A). The inventors validated this dual-indexing approach by processing single HEK 293T cells with 10 CCs and 12 well-barcodes.

Initially, the inventors calculated the number of reads retrieved for each cell, well, and PC and observed that some of the barcodes yielded varying number of reads (FIG. 9B).

When the inventors assessed the UMIs identified per barcode as a proxy for sensitivity (FIG. 9C), the inventors observed that three CCs (Lid7, Lid14, and Lid15) and two WCs (S501, S504) had decreased UMI counts, while all PCs were comparable (FIG. 9C), suggesting that overall library quality is robust across single cells and variance in data quality was due to quality differences of the utilized primers. Overall, our analysis suggested that our novel system offers an even cell quality with high library complexity, with some dependency on primer quality.

Having shown the utility of our dual-barcoding chemistry for scRNA-seq application, the inventors further validated our approach by distinguishing biological states, and to further demonstrate that deterministic barcoding could be used to freely multiplex samples.

Therefore, the inventors used RAW 264.7 mouse macrophages and generated two distinct cell states by Lipopolysaccharide (LPS) stimulation. The inventors processed untreated control cells (Ctrl) and LPS-treated cells (LPS) in alternating fashion, so each CC was associated with a specified cell-state (FIG. 10A). In total the inventors processed 384 cells with six CCs, of which the inventors were able to identify 76%, or 283 cells, matching the observed encapsulation efficiency of approximately 80%, i.e. the percentage of droplets containing a cell during the experiment, underscoring that RT in droplets is reliable and that minimal loss occurs through-out the complete workstream. From the quality-filtered cells the inventors assembled a dimensionality reduced global scaffold (FIG. 10B) and identified two distinct cell distinct clusters. One cluster showed high expression of immune response genes (FIG. 10C). To confirm whether CCs and states matched, the inventors bioinformatically stratified the cells in the global scaffold by CC. Indeed, the inventors found that the vast majority of cells associated to each CC was in the cell state's expected cluster (FIG. 10D).

In sum, this state change experiment confirmed that our fully deterministic scRNA-seq approach was capable of faithfully capturing biological states and that samples can be multiplexed natively on our system, a critical advantage of deterministic barcoding.

The general principle of deterministically barcoding the molecules of an encapsulated entity are also applicable to DNA molecules, including but not exclusively ChIP-seq, TF-seq and ATAC-seq,^{29,30} pertaining the following key modifications to the process described above. 1) Loading cells with Tn5 pre-loaded with a DNA tag, which is not S5 or S7. 2) Deterministically encapsulating Tn5 pre-loaded cells with a PCR solution containing the cell specific primers. 3) Performing CC-PCR within droplets which adds the CC via the overhang appended by the initial Tn5 loaded to the cellular DNA. 4) Merging the droplets containing cell-specific barcoded molecules, with a secondary WC-PCR solution that contains the WC primer on a per well basis (FIG. 11). The described process is also applicable to ChIP-seq and TF-seq with a prepended processing step, that labels the mark of choice based on antibody-based labelling.

### Deterministic Barcoding Enables Processing of Bulk RNA-Seq Per Multi-Well Unit

The inventors also successfully utilized the dual-barcoding approach for pooled samples, obtaining bulk RNA-seq libraries from five HEK 293T cells, where the RT was performed for each cell in a single nanoliter-droplet and subsequent well-coding for the pooled 5 cells, recovering all eight samples per PC comprising or consisting, for example, of 5 cells per sample with similar number of sequenced reads per sample (FIG. 12A). UMI detection was equivalent across WCs (FIG. 12B). Furthermore, the inventors were able to use only one barcode to rapidly perform RT for each of the samples and obtain libraries with high quality and minor variation across the samples, underscored by the minor variations for the Top30 genes, percent of mitochondrial reads, heat-shock proteins and ribosomal protein reads (FIG. 12C-D). Thereby the inventors are able to consistently multiplex samples using as little as five cells as input to obtain robust 3′RNA-seq profiles.

### ANNEXES

### Annex 1: Droplet Encapsulation and Assembly of Consortia

Barcoded reaction buffer (RB) was introduced into the RB inlet, and cells in cell in the cell inlet. Both solutions were flown into the chip for approx. 30 sec at approx. 200 mbar pressure. For deterministic encapsulation of droplets and evacuation sorting of same droplet the following process was implemented on the system:
a) Cell stopping:
   1) Primary cell detection
   2) Close cell valve
   3) Over pressure cell inlet
   4) Secondary cell detection
   5) Depressure cell inlet
b) Droplet generation:
   1) Open oil valve to clean dropleting area
   2) close oil valve
   3) Pressurization of droplet valve
   4) Open sample valve, close waste
   5) Open of the oil valve
   6) Open waste valve and close sample valve
c) Droplet evacuation:
   1) Open off-chip oil solenoid
   2) flush approximately 1 second
   3) Close off-chip solenoid
d) Prepare encapsulation area for next encapsulation:
   1) Replenish RB-solution by over pressurization of RB valve
   2) Retract capillary
   3) Move plate to next well
   4) Insert capillary

Once each unit of the multi-well is loaded with a droplet containing the barcoded reaction buffer, the reaction buffer loading pipette tip is removed from the chip, a new gel loading pipette tip inserted, filled with 20 μL of dH2O, and the channel washed. Then the next barcode was loaded by adding RB-solution and primed for 30 seconds at 200 mbar.

### Annex 2: Multi-Modal Imaging of Cells

Detection: 40×-objective was used in this setup, the detection image required de-magnification to visualize the necessary FOV for fast and automated cell detection and accurate placement. The de-magnification of the detection image was achieved by aligning two lenses (f=75 mm followed by f=25 mm) subsequent to the objective instead of using a tube lens, resulting in a final 2× magnification, which is slightly lower than the calculated theoretical value of 2.4×. The process for detection and stopping at the imaging area was as follows:
1) Primary cell detection
2) Close cell valve
3) Over pressure cell inlet
4) Secondary cell detection
5) Depressure cell inlet

For high-resolution imaging, the inventors used a tube lens with a focal length of 200 mm to achieve the 40×-magnification, for which the objective is designed for. However, adding the ETL with the 4f-relay system (comprising or consisting, for example of two lenses with different focal lengths (200 mm and 100 mm)) resulted in a de-magnification. Since the resulting FOV covered a larger area than the ROI, the inventors compensated for the de-magnification by adding a 2×-beam-expander. Although placing the cell in the ROI is relatively accurate, it is not possible to place the cell at a fixed z-position. Therefore, if the cell is not positioned correctly, it will be out of focus. By integrating an ETL, the inventors enabled z-axial-scanning through the channel, and thus permitted to acquire at least one focused image of the target cell. To avoid magnification variations and still enable a large z-axial scanning range the inventors inserted the ETL along with a 4f-relay system. It should be noted that the inventors cannot distinguish between positioning the ETL after or between the 4f-relay lenses, since the first relay lens has the same focal length as the tube lens (f=200 mm), which are, hence, interchangeable. The relay system consisted of f=200 mm and f=100 mm lenses, resulting in a scanning range of about 60 μm. The images were acquired as follows upon stopping the cell in the imaging area:
1) Bright-field image per focal plane with a total of 8 planes imaged
2) Epi-fluorescence image per focal plane with a total of 8 planes imaged

### Annex 3: Nuclei Isolation and Processing on IRIS

a) Nuclei isolation
   prepare homogenization and resuspension buffer
   Thaw samples from −80° C. on ice if using frozen samples and add 100 ul “homogenization buffer”
   Add 900 ul homogenization buffer, and transfer 1000 ul homogenized sample
   Release nuclei by 15-20 resuspensions
      Keep on ice. Avoid foam.
   filter 1000 ul sample through 5 ml cell strainer (35 um)
   filter 2^{nd} time using 40 um FlowMi (Merck, #BAH136800040) strainer into 1.5 ml Eppi tube.
   Centrifuge for 10 min at 1000 g at 4° C. and discard the supernatant
      Do not disturb the pellet (sometimes invisible).
   resuspend in 1000 ul “Resuspension buffer”.
   filter sample using 40 um FlowMi strainer into 1.5 ml Eppi and deposit on ice
   count nuclei
b) IRIS processing
   resuspend nuclei at concentration of 10'000-30'000 cell/ml in PBS (840 ul PBS+10 ul RNAse Inhibitor (NEB, #M0314L)+150 ul Optiprep (Sigma-Aldrich, #D1556))
   keep on ice
   load 100 ul of nuclei suspension into western tips
1) Homogenization buffer (make fresh and keep on ice) for 10 ml:

| 10 ml RT H2O (nuclease free) | | |
|---|---|---|
| 0.856 g Sucrose (Sigma-Aldrich, #84097-250G) | 250 | mM |
| 50 ul 2M Tris PH 8.0 (Sigma-Aldrich, #T2944-100ML) | 10 | um |
| 250 ul 1M KCl (Sigma-Aldrich, #7447-40-7) | 25 | mM |
| 50 ul 1M MgCl2 (Sigma-Alrdrich, #M1028-10X1ML) | 5 | mM |
| 50 ul 20% Triton-x 100 (Sigma-Aldrich, #T8787) | 0.1% | |
| 25 ul RNase Inhibitor (NEB, #M0314L) | 0.25% | |
| 100 ul 100x protease inhibitor | 1x | |
| (Sigma-Aldrich, #11206893001) | | |
| 10 ul −20° C. 100 mM DTT | 0.1 | mM |
| (AppliChem, #A2948, 0005) | | |

2) Resuspension buffer for 10 ml:
   9.5 ml 1× PBS (Gibco, #10010023)
   0.5 ml 10% BSA (final 0.5%, Sigma-Aldrich, #A9418)
   25 ul RNase Inhibitor (NEB, #M0314L)

### Annex 4: Reverse Transcription, Dual-Indexing of mRNA, cDNA Amplification

The reaction buffer contained, the following components:

| | | | Concentration in | |
|---|---|---|---|---|
| | Manufacturer | Catalogue # | reaction buffer | |
| Triton X-100 | Sigma-Aldrich | T8787 | 1% | w/v |
| dNTPs | Sigma-Aldrich | DNTP100-1KT | 1 | mM |
| RT buffer | Thermo Scientific | EP0752(THE) | 1.72fold | |
| Tris-HCl | Sigma-Aldrich | T2944-100ML | 0.1M | |
| DTT | AppliChem | A2948, 0005 | 6.8 | mM |
| MgCl2 | Sigma-Aldrich | M1028-10X1ML | 10 | mM |
| RNAse | NEB | M0314L | 1 | unit/μl |
| inhibitor | | | | |
| Maxima | Thermo Scientific | EP0752(THE) | 10 | units/μl |
| Enzyme | | | | |

| | | | |
|---|---|---|---|
| Optiprep | Sigma-Aldrich | D1556 | 15% |

Once the desired number and composition of droplet consortia was accomplished, multi-well plates were sealed with Thermowell Sealing Tape (Corning, #6570).

Three distinct reverse transcription primers were utilized (all IDT).

### I) Full-Length Transcript Non-Barcoded Primers

See Annex 7

### II) Single Barcode Chemistry Primers

See Annex 7

### III) Dual Barcode Chemistry Primers

Version 1

See Annex 7
Version 2

See Annex 7

The sealed plates were incubated for 60 min at 50 C on a Biometra (Analytic Jena) thermocycler. Subsequently, multi-well plates were placed in the Droplet merging device, and merged for 10 sec at 1.3 kV. Three distinct protocols were employed for PCR amplification:

### I) Full-Length Transcript Non-Barcoded

9 μL PCR solution containing 1× KAPA HiFi HotStart (Roche, #07958935001)
0.64 μM SMART-PCR
PCR for: 3 minutes at 98° C.,
   determined number cycles with: 20 sec 98° C., 15 sec 67° C., 6 min 72° C. 10 min 72° C.
Amplified cDNA was purified for each well individually.

### II) Single Barcode Chemistry

9 μL PCR solution containing 1× KAPA HiFi HotStart (Roche, #07958935001)
0.64 μM SMART-PCR oligonucleotide
PCR for: 3 min at 98° C.
   determined number cycles with: 20 sec 98° C., 30 sec 67° C., 4 min 68° C. 10 min 72° C.
   Amplified cDNA was purified for each well individually.

### III) Dual-Barcode Chemistry (Version1)

Dual barcode biochemistry required to perform two separate PCRs. On for pre-post-amplification and wellcoding and one for amplification of the full library.

### Pre-Amplification:

9 μl PCR solution containing 1× KAPA HiFi HotStart (Roche, #07958935001)
0.15 μM wellcode primer
0.64 μM SMART-PCR oligonucleotide.
For each well that was pooled later in the process a well specific barcode was added. In the experiments displayed in this study the inventors used a total of 12 well code primers.
PCR for: 3 min at 98° C.
   5× cycles: 15 sec 98° C., 45 sec 65° C., 4 min 68° C.
   10 min 72° C.
Wells with different wellcodes were pooled, and the pooled consortia PCR purified with

### Post-Amplification:

0.7× CleanPCR magnetic beads and eluted in 15 ul of dH2O.
0.15 μM P5 primer
0.64 μM SMART-PCR oligonucleotide.
PCR for: 3 min at 95° C.
   determined number cycles with: 15 sec 98° C., 30 sec 65° C., 4 min at 68° C. 10 min 72° C.

The number of PCR cycles was determined experimentally for each input type (e.g. RAW, HEK 293T, neural nuclei, mRNA). Cycle numbers ranged between 10-24 cycles. PCR amplification was performed on a Biometra (Analytik Jena).

For Dual-barcode chemistry (Version2) the annealing temperature for the Pre-amplification is at 57° C. and for Post-amplification at 60° C.

### Annex 5: Tagmentation, Dual-Indexing of DNA Per Cell, Sequencing Library Preparation

For PCR purification CleanPCR magnetic beads (CleanNA, #CPCR-0050) were used at a 0.8× ratio according to the manufacturer protocol. The purified cDNA was quantified with a Qubit HS-DNA assay, and cDNA size distribution validated on a Fragment analyzer (Agilent). The quality-controlled cDNA was tagmented using Tn5 produced in-house,⁴⁵ loaded with s7 Tn5 sequence. The fragments were PCR amplified with P5-TSO hybrid primer for single barcoding and i7 indexing primers (PC) and P5 sequence primers for dual barcoding with the following PCR protocol:
3 min 72° C., 30 sec 98° C.
16× cycles: 10 sec 98° C., 30 sec 63° C., 1 min 72° C.
5 min at 72° C.

Tagmentation was followed by 2 rounds of size selection at 0.7× ratio. The yield of the purified products were quantified using Qubit, and the size distribution validated on a Fragment analyzer.

### Annex 6: Primers Used

All primers were ordered from IDT (Integrated DNA Technologies)

bioTSO|
(SEQ ID NO 1)
5′-/5Biosg/AAGCAGTGGTATCAACGCAGAGTACATrGrG+G-3′
biotin-oligo-dT30VN|
(SEQ ID NO 2)
5′-/5Biosg/AAGCAGTGGTATCAACGCAGAGTAC(30xT)VN-3′
P5-TSO hybrid|
(SEQ ID NO 3)
5′-AATGATACGGCGACCACCGAGATCTACACGCCTGTCCGCGGAAGCAG
TGGTATCAACGCAGAGT*A*C-3′
bioTSO|
(SEQ ID NO 4)
5′-/5Biosg/AAGCAGTGGTATCAACGCAGAGTGAATrGrGrG-3′
TSO-PCR|
(SEQ ID NO 5)
5′-AAGCAGTGGTATCAACGCAGAGT-3′
S7
(SEQ ID NO 6)
5′-GTCTCGTGGGCTCGGAGATGTGTATAAGAGACAG-3′
S5
(SEQ ID NO 7)
5′-TCGTCGGCAGCGTCAGATGTGTATAAGAGACAG-3′
i7|
(SEQ ID NO 8)
5′-CAAGCAGAAGACGGCATACGAGAT[8bp_i7]GTCTCGTGGGCTCG
G-3′ [8bp_i7] commercially available and custom
sequences
i5|
(SEQ ID NO 9)
5′-AATGATACGGCGACCACCGAGATCTACAC[8bp_i5]TCGTCGGCAG
CGTC-3′ [8bp_i5] commercially available and custom
sequences (see below) at WC_V2
P5|
(SEQ ID NO 10)
5′-AATGATACGGCGACCACCGAGATCTACAC-3′
Cell-barcode Version1|
(SEQ ID NO 11)
5′-/5Biosg/TCGTCGGCAGCGTCAGATGTGTATAAGAGACAG
[CC_V1]NNNNNNNNNNVVVVV(30xT)VN-3′
[CC_V1] Hamming distance 4
(SEQ ID NO 12)
TACCTCTCGG
(SEQ ID NO 13)
CTGCCATCAC
(SEQ ID NO 14)
AACTCGTTCG
(SEQ ID NO 15)
TAAGTGCTCG
(SEQ ID NO 16)
CCATATCGGA
(SEQ ID NO 17)
CTAACACAGA
(SEQ ID NO 18)
CATTGCCTGG
(SEQ ID NO 19)
AATGAGAGAG
(SEQ ID NO 20)
TTCCAATACG
(SEQ ID NO 21)
ACAGTTAGCA
(SEQ ID NO 22)
ACCAATGCGG
(SEQ ID NO 23)
AGATCCGCTC
(SEQ ID NO 24)
ATTCGCGTCG
(SEQ ID NO 25)
CACACCGAGA
(SEQ ID NO 26)
CGATGCACAC
Well-barcode Version1|
(SEQ ID NO 27)
5′-AATGATACGGCGACCACCGAGATCTACAC[WC_V1]TCGTCGGCAGC
GTCAGATGTG-3′
[WC_V1] commercially available and custom
sequences (see below) at WC_V2
Cell-barcode Version2|
(SEQ ID NO 28)
5′-/5Biosg/CGTCAGATGTGTATAAGAGACAG[CC_V2]NNNNNNVVV
(30xT)VN-3′
[CC_V2] Hamming distance 4
TACCTCT ACAAGCG ACGCTGT CAACAGG CTTCCGC CATGACC
AGTCGCT CGAGCAC GTAGACG TGATCGG TGTGTCG GATCGTC
AAACCCC AACACGG AAGCGAG ACAGAGC ACCCACA ACCGCAT
ACCTGTC ACTCCTG AGCATCC AGCGATG AGTGCGA ATGTCCG
CACAGAC CACCCTA CAGACCT CAGTTGC CCAACGA CCACGAT
CCAGTTG CCCTAAG CCGAATC CCGTGCA CGACTCA CGCAAGT
CGTAGTG CTCGTGA GAAGCGT GACGTAG GCATTCC GCGACAG
GTCACCA GTCCAAC GTGCCTT TCTACCC TGCCGAA TTCGCTC
TTGCGCC
Well-barcode Version2|
(SEQ ID NO 29)
5′-AATGATACGGCGACCACCGAGATCTACAC[WC_V2]TCGTCGGCAGC
GTCAGATGTGTATAAGAGACAG-3′
[WC_V2] Hamming distance 3
TAGATCGC CTCTCTAT TATCCTCT AGAGTAGA GTAAGGAG
ACTGCATA AAGGAGTA CTAAGCCT CGTCTAAT TCTCTCCG
TCGACTAG TTCTAGCT CCTAGAGT GCGTAAGA CTATTAAG
AAGGCTAT GAGCCTTA TTATGCGA TGTCTCGC TCGTGAGC
TAGCGAGG GTCAGCCG GCTCAACG GATGTCCG GACTGCAC
CTGCAGCC CTACTCCG CGAGTGCA CATCCCGC ATGCCACC
AAACCCCC AAACGCGG AAAGCGCG AACACCCG AACAGCGC
AACCAGCC AACCCAGC AACCCGAG AACCGACG AACGACGG
AACGCCAC AAGACGCC AAGCACCG AAGCCCGA AAGCGCAC
ACAACCGC ACAAGCCG ACACAGCG ACACCAGG ACACCGAC
ACACGACC ACAGACCC ACAGCCAG ACCACACC ACCACGTG
ACCAGAGG ACCCACAC ACCCATGG ACCCCCCA ACCCCTTC
ACCCGCGT ACCCTCTG ACCCTGCT ACCGAACG ACCGAGTC
ACCGCAGT ACCGCGAA ACCGTCGA ACCGTTCC ACCTCCGG
ACCTGCCC ACGAACGG ACGACCCT ACGAGCTC ACGATGCG
ACGCAAGC ACGCCGTA ACGCCTCG ACGCGAAG ACGCGTGA
ACGCTCCC ACGTAGCC ACGTCCAC ACTGCGCC ACTGCTGG
AGACACGC AGACCCTG AGACCGCA AGACGCCT AGAGCACC
AGAGCCGT AGAGTCCG AGCAACCC AGCACCGA AGCACGAC
AGCAGCAG

### Annex 7: Sequencing and Analysis Strategy

Illumina NextSeq 500 platform using High Output v2 kit (75 cycles) (Illumina, #FC-404-2005). The library loading concentration was between 1.5 to 2.2 μM and sequencing configuration as following for version1 biochemistry: R1 25 c/i5 8c/i7 8c/R2 50 c. The sample reads base calling and demultiplexing was carried out using bcl2fastq by i5 and i7. The fastq files per i5&i7 were processed with STAR version 2.7 with the whitelist containing the respective CCs.⁴⁶ Mapping was carried out on the respective genomes (GRCh38.100, mm10) or their assemblies or including the ERCC sequences.⁴⁷ Count matrices per well were further processed using R software version 3.2.0 and Seurat version 3.2.0.⁴⁸

### REFERENCES

1. Tang, F. et al. mRNA-Seq whole-transcriptome analysis of a single cell. Nat. Methods 6, 377-382 (2009).
2. Hashimshony, T., Wagner, F., Sher, N. & Yanai, I. CEL-Seq: Single-Cell RNA-Seq by Multiplexed Linear Amplification. Cell Rep. 2, 666-673 (2012).
3. Islam, S. et al. Characterization of the single-cell transcriptional landscape by highly multiplex RNA-seq. Genome Res. 21, 1160-1167 (2011).
4. Pollen, A. A. et al. Low-coverage single-cell mRNA sequencing reveals cellular heterogeneity and activated signaling pathways in developing cerebral cortex. Nat. Biotechnol. 32, 1053-1058 (2014).
5. Kolodziejczyk, A. A., Kim, J. K., Svensson, V., Marioni, J. C. & Teichmann, S. A. The Technology and Biology of Single-Cell RNA Sequencing. Molecular Cell 58, 610-620 (2015).
6. Lane, K. et al. Measuring Signaling and RNA-Seq in the Same Cell Links Gene Expression to Dynamic Patterns of NF-κB Activation. Cell Syst. 4, 458-469.e5 (2017).
7. Macosko, E. Z. Z. et al. Highly parallel genome-wide expression profiling of individual cells using nanoliter-droplets. Cell 161, 1202-1214 (2015).
8. Zheng, G. X. Y. et al. Massively parallel digital transcriptional profiling of single cells. Nat. Commun. 8, 14049 (2017).
9. Klein, A. M. et al. Droplet barcoding for single-cell transcriptomics applied to embryonic stem cells. Cell 161, 1187-1201 (2015).
10. Han, X. et al. Mapping the Mouse Cell Atlas by Microwell-Seq. Cell 172, 1091-1107.e17 (2018).
11. Gierahn, T. M. et al. Seq-Well: Portable, low-cost ma sequencing of single cells at high throughput. Nat. Methods 14, 395-398 (2017).
12. Goldstein, L. D. et al. Massively parallel nanowell-based single-cell gene expression profiling. BMC Genomics 18, 519 (2017).
13. Lun, A. T. L. et al. EmptyDrops: Distinguishing cells from empty droplets in droplet-based single-cell RNA sequencing data. Genome Biol. 20, 63 (2019).
14. DePasquale, E. A. K. et al. DoubletDecon: Deconvoluting Doublets from Single-Cell RNA-Sequencing Data. Cell Rep. 29, 1718-1727.e8 (2019).
15. McGinnis, C. S., Murrow, L. M. & Gartner, Z. J. DoubletFinder: Doublet Detection in Single-Cell RNA Sequencing Data Using Artificial Nearest Neighbors. Cell Syst. 8, 329-337.e4 (2019).
16. Ding, J. et al. Systematic comparison of single-cell and single-nucleus RNA-sequencing methods. Nat. Biotechnol. 38, 737-746 (2020).
17. Stoeckius, M. et al. Cell Hashing with barcoded antibodies enables multiplexing and doublet detection for single cell genomics. Genome Biol. 19, 224 (2018).
18. McGinnis, C. S. et al. MULTI-seq: sample multiplexing for single-cell RNA sequencing using lipid-tagged indices. Nat. Methods 16, 619-626 (2019).
19. Stoeckius, M. et al. Simultaneous epitope and transcriptome measurement in single cells. Nat. Methods 14, 865-868 (2017).
20. Zhang, X. et al. Comparative Analysis of Droplet-Based Ultra-High-Throughput Single-Cell RNA-Seq Systems. Mol. Cell 73, 130-142.e5 (2019).
21. Liu, Z. et al. Integrating single-cell RNA-seq and imaging with SCOPE-seq2. Sci. Rep. 10, 1-15 (2020).
22. Bush, E. C. et al. PLATE-Seq for genome-wide regulatory network analysis of high-throughput screens. Nat. Commun. 8, (2017).
23. Alpern, D. et al. BRB-seq: ultra-affordable high-throughput transcriptomics enabled by bulk RNA barcoding and sequencing. Genome Biol. 20, 71 (2019).
24. Bues, J. et al. Deterministic scRNA-seq of individual intestinal organoids reveals new subtypes and coexisting distinct stem cell pools. bioRxiv 2020.05.19.103812 (2020). doi:10.1101/2020.05.19.103812
25. Picelli, S. et al. Full-length RNA-seq from single cells using Smart-seq2. Nat. Protoc. 9, 171-181 (2014).
26. Chen, W., Gardeux, V., Meireles-Filho, A. & Deplancke, B. Profiling of Single-Cell Transcriptomes. in Current Protocols in Mouse Biology 7, 145-175 (John Wiley & Sons, Inc., 2017).
27. Bagnoli, J. W. et al. mcSCRB-seq: sensitive and powerful single-cell RNA sequencing. bioRxiv 188367 (2017). doi:10.1101/188367
28. Chen, X., Miragaia, R. J., Natarajan, K. N. & Teichmann, S. A. A rapid and robust method for single cell chromatin accessibility profiling. Nat. Commun. 9, 1-9 (2018).
29. Buenrostro, J. D., Wu, B., Chang, H. Y. & Greenleaf, W. J. ATAC-seq: A Method for Assaying Chromatin Accessibility Genome-Wide. Curr. Protoc. Mol. Biol. 109, 21.29.1-9 (2015).
30. Skene, P. J. & Henikoff, S. An efficient targeted nuclease strategy for high-resolution mapping of DNA binding sites. Elife 6, (2017).
31. Howell, J., Hammarton, T. C., Altmann, Y. & Jimenez, M. High-speed particle detection and tracking in microfluidic devices using event-based sensing. Lab Chip (2020). doi:10.1039/DOLC00556H
32. Sesen, M. & Whyte, G. Image-Based Single Cell Sorting Automation in Droplet Microfluidics. Sci. Rep. 10, 1-14 (2020).
33. [DE], C. C. [DE]; M. C. A. [DE]; N. L. [DE]; U. R. CELL BARCODING IN MICROFLUIDICS. Espacenet B01L3/00, (2018).
34. Rotem, A. et al. High-Throughput Single-Cell Labeling (Hi-SCL) for RNA-Seq Using Drop-Based Microfluidics. PLOS One 10, e0116328 (2015).
35. Rotem, A. et al. Single-cell ChIP-seq reveals cell subpopulations defined by chromatin state. Nat. Biotechnol. 33, 1165-1172 (2015).
36. Eduati, F. et al. A microfluidics platform for combinatorial drug screening on cancer biopsies. Nat. Commun. 9, 1-13 (2018).
37. [US], B. K. [US]; B. R. [US]; H. C. [US]; L. B. [US]; M. A. [US]; P. P. [US]; P. K. [US]; P. A. D. [US]; R. L. M. [US]; S.-L. M. [US]; W. T. D. [. METHODS AND SYSTEMS FOR DROPLET-BASED SINGLE CELL BARCODING. Espacenet C12N15/09; (2018).
38. Yuan, J., Sheng, J. & Sims, P. A. SCOPE-Seq: a scalable technology for linking live cell imaging and single-cell RNA sequencing. Genome Biol. 19, 227 (2018).
39. [CA], H. A. [US]; J. V. [US]; S. A. APPARATUS FOR HIGH THROUGHPUT CHEMICAL REACTIONS. Espacenet AU20082095, (2008).
40. HUDA AMJAD [US]; JOSEPH VICTOR [US]; SHIVJI ALNOOR [US]; ZHOU JIE [US]. Thermo-controllable high-density chips for multiplex analyses. Espacenet EP1758981A, (2004).
41. DONG WESLEY B [US]; DUNNE JUDE [US]; GRISWOLD BRADLEY L [US]; HUSAIN SYED A [US]; KOTTURI GOPAUL [US]; LIN PHILIP [US]; MOTI ROBERT [US]. Extraction of restrained liquid from wells. Espacenet AU20133422, (2012).
42. [US], C. T. H. [US]; C. C. G. [US]; W. X. [US]; W. J. A. A. Single cell nucleic acids for high-throughput studies. Espacenet CA2976681A, (2015).
43. [US], A. W. [US]; A. C. [US]; C. S. H. [US]; M. Z. [US]; M. J. M. [US]; N. G. [US]; P. P. SYSTEMS, APPARATUSES, AND METHODS FOR CELL SORTING AND FLOW CYTOMETRY. Espacenet CN10792338, (2015).
44. Nitta, N. et al. Intelligent Image-Activated Cell Sorting. Cell 0,
45. Picelli, S. et al. Tn5 transposase and tagmentation procedures for massively scaled sequencing projects. Genome Res. 24, 2033-2040 (2014).
46. Dobin, A. & Gingeras, T. R. Mapping RNA-seq Reads with STAR. Curr. Protoc. Bioinforma. 51, 11.14.1-19 (2015).
47. Jiang, L. et al. Synthetic spike-in standards for RNA-seq experiments. Genome Res. 21, 1543-1551 (2011).
48. Butler, A., Hoffman, P., Smibert, P., Papalexi, E. & Satija, R. Integrating single-cell transcriptomic data across different conditions, technologies, and species. Nat. Biotechnol. 36, 411-420 (2018).

1. A system for phenotypical profiling of at least one object and deterministic nanoliter-droplet encapsulation, the system comprising:
sample supplying means comprising at least one sample reservoir configured to contain at least one object dispersed in a sample buffer;
buffer supplying means comprising at least one buffer reservoir configured to contain a primary reaction buffer;
a microfluidic chip comprising:
a first imaging chamber;
a first microfluidic channel for transporting the at least one object from the sample supplying means to the first imaging chamber;
an oil inlet for introducing an oil that supports droplet formation in the microfluidic chip or a droplet forming substance inlet for introducing a droplet forming substance into the microfluidic chip;
an encapsulation area or structure in which the at least one object is encapsulated with a quantity of the primary reaction buffer by the formed droplet;
a second microfluidic channel for transporting the primary reaction buffer from the buffer supplying means to the encapsulation area or structure;
an oil supporting droplet formation microchannel or droplet forming substance microchannel connected to the encapsulation area to place the at least one object and the primary reaction buffer in direct contact with the oil that supports droplet formation or the droplet forming substance;
a droplet microchannel or tubing for transporting the droplet;
detection means configured to detect the passage of the at least one object through the first imaging chamber;
at least one valve configured to stop the flow of the sample buffer when the detection means detect the passage of the at least one object through the first imaging chamber;
phenotypical assessing means configured to assess the phenotype of the at least one object when the flow of the sample buffer is stopped by the at least one valve and the at least one object is at an object stopping site;
a droplet deposition means configured to deposit the droplet in a well or in a well of a multi-well plate and comprising an outlet capillary connected to the droplet microchannel or the tubing.

2. The system according to claim 1, wherein the at least one object is a cell, a cellular entity or a cellular compartment.

3. The system according to claim 1, wherein the primary reaction buffer is configured to perform a first reaction and comprises an enzyme and/or a biochemistry of choice and/or a culture medium and/or growth matrices and/or a reverse transcriptase and/or a hyperactive transposase and/or a first molecular barcode such as a phenotype barcode.

4. The system according to claim 1, wherein the buffer supplying means comprises at least one secondary buffer reservoir containing a placement buffer configured to instigate a biological reaction of the at least one object and/or enable an imaging-based assessment of the biological reaction.

5. The system according to claim 1, wherein the at least one object is positioned at the object stopping site by displacement of the sample buffer and/or the placement buffer and/or the primary reaction buffer.

6. The system according to claim 1, wherein the well or the wells of a multi-well plate are pre-loaded with stuffer droplets comprising the primary reaction buffer and/or a secondary reaction buffer to perform a secondary reaction inside the well or the wells of a multi-well plate, the stuffer droplets being configured to be merged with the droplet comprising the least one object and the primary buffer.

7. The system according to claim 1, wherein the well or the wells of a multi-well plate comprise a second molecular barcode, such as a well molecular barcode.

8. The system according to claim 1, comprising a second imaging chamber fluidically connected to the first imaging chamber, the second imaging chamber comprising an object stopping site.

9. The system according to claim 1, wherein the at least one valve is configured to be over-pressured to generate a leak flow of the sample buffer and/or the placement buffer and/or the primary reaction buffer until the at least one object reaches the object stopping site or the second imaging chamber.

10. The system according to claim 1, wherein the detection means and the phenotypical assessing means comprise a microscope comprising a dual-camera objective imaging and detection system, and/or an tunable lens to adjust the focal plane, and/or a laser excitation diode for epi-fluorescence imaging.

11. The system according to claim 1, wherein the first and/or second imaging chambers and/or the encapsulation area and/or the inlets comprises rectangular channel structures and wherein an area around the at least one valve are non-rectangular channel structures.

12. The system according to claim 1, wherein the sample supplying means comprise several reservoirs each containing at least one object dispersed in a sample buffer.

13. The system according to claim 1, wherein the buffer supplying means comprise several reservoirs, containing several primary buffers having different compositions and/or phenotype barcode identifiers.

14. The system according to claim 1, wherein the droplet deposition means is configured to deposit the droplet in a specific well of a multi-well plate, the droplet deposition means comprising plate displacing means configured to displace horizontally and/or vertically the multi-well plate relative to the outlet capillary.

15. The system according to claim 1, wherein the system comprises a processor configured to obtain and digitally store detection and phenotype data of at least one object from the detection and phenotypical assessing means and to link the detection and phenotype data with the phenotype and/or molecular barcode identifier encapsulated with the at least one object and the well in which the droplet is deposited.

16. The system according to claim 1, wherein the processor is further configured to operate an image-based selection process prior or previous to the at least one object encapsulation to deposit the droplet in a specific well depending on the phenotype of the encapsulated object and/or to discard the unwanted object or droplet comprising the unwanted object with a discarding valve configured to open to discard unwanted object.

17. A method of operating a system according to claim 1 for phenotypical profiling of at least one object and deterministic nanoliter-droplet encapsulation, the method comprising the steps of:
Introducing the at least one object from the sample supplying means into the first imaging chamber through the first microfluidic channel;
Stopping the flow of the sample buffer when the detection means detect the passage of the at least one object through the first imaging chamber;
Assessing the phenotype of the at least one object when the flow of the sample buffer is stopped and the at least one object is at an object stopping site;
Introducing the primary reaction buffer from the buffer supplying means into the microfluidic chip through the second microfluidic channel;
Introducing the oil that supports droplet formation into the microfluidic chip or the droplet forming substance into the microfluidic chip through the oil inlet or the droplet forming substance inlet;
Transporting the at least one object and the primary reaction buffer to the encapsulation area or structure for encapsulation by the droplet;
Transporting the droplet to the droplet deposition means through the droplet microchannel or tubing for deposition of the droplet in a well or in a well of a multi-well plate.

18. A defined group of droplets placed in one well, each containing at least one object, with each droplet containing one defined primary buffer and/or phenotype barcode identifier.
